(19) United States
(12) Patent Application Publication
(10) Pub. No.: US 2017/0173134 A1
(43) Pub. Date: Jun. 22, 2017

(54) IMMUNOMODULATOR FOR THE TREATMENT OF CANCEROUS AND PRECANCEROUS LESIONS IN THE EPITHELIAL TISSUE LINING SURFACES INSIDE OR OUTSIDE BODY ORGANS

(71) Applicant: Iseu da Silva NUNES, Campinas (BR)

(72) Inventor: Iseu da Silva NUNES, Campinas (BR)

(21) Appl. No.: 15/443,564

(22) Filed: Feb. 27, 2017

Related U.S. Application Data
(63) Continuation-in-part of application No. 14/444,436, filed on Jul. 28, 2014.
Application No. 14/444,436 is a continuation-in-part of application No. 14/357,490, now abandoned.

(51) Int. Cl. A61K 39/00; A61K 45/06; A61K 47/06; A61K 47/02; A61K 9/00; A61K 47/34

(52) U.S. Cl. CPC A61K 39/0011; A61K 9/0014; A61K 47/34; A61K 47/06; A61K 47/02; A61K 45/06; A61K 2039/585; A61K 2039/54

(57) ABSTRACT

“IMMUNOMODULATOR FOR TOPICAL TREATMENT OF MALIGNANT AND PREMALIGNANT LESIONS IN THE EPITHELIAL TISSUE LINING INSIDE AND OUTSIDE SURFACES OF THE BODY”, the present invention provides a novel method of treatment for malignant and premalignant lesions in the epithelial tissue lining inside and outside surfaces, comprising the topical use of the immunomodulator, either alone or in association or combination with other drug and non-drug treatments and pharmaceutical compositions for its use.

### CROSS REFERENCE TO RELATED APPLICATIONS

This application is a continuation in part of co-pending U.S. Ser. No. 14/444,436, filed 28 Jul. 2014, and for which is claimed under 35 U.S.C. §120, which claims priority to Application PCT/BR2011/000419 filed in Brazil on Nov. 9, 2011, the entire contents of all of which are hereby incorporated by reference.

The present patent application concerns a method of topical treatment of malignant or premalignant lesions in the epithelial tissue lining surfaces outside and inside the body, using an immunomodulator, which can be used alone or in combination or association with other drugs or therapies as well as inventive pharmaceutical formulations for its use in subjects in need thereof.

### EPITHELIAL TISSUE AND CANCER

Epithelial Tissue—General Description

The epithelial tissue is a special kind of cell structure that covers the entire outer surface of the body or skin, and all internal body cavities, such as the pleural, pericardial and peritoneal cavities. It also forms the lining of the heart, blood and lymphatic vessels, digestive tract and genitourinary tract.

Consists almost exclusively of juxtaposed cells, with very little of no intercellular substance between them. The cells adhere to each other by means of intercellular junctions or by proteins on the cell membrane.

The lining epithelial tissue does not have its own vascular system, and, thus, nutrients for the cells are obtained from capillaries in adjacent connective tissue via diffusion.

The epithelial tissue performs many important functions, such as: protection and covering (e.g. skin), secretion (e.g. stomach), mixed functions of secretion and absorption (e.g. intestine), waterproofing (e.g. urinary bladder).

Finally, it should be noted that, just as in any other tissue, malignant tumors can originate in the epithelial tissue, and it is noteworthy that many malignant tumors in humans and animals originate or are located in this particular type of tissue.

This fact is not surprising, considering that the epithelial tissue, due to its characteristics and functions, has cells that must be constantly renewed and are almost continuously exposed to aggressive external agents such as solar radiations and potentially carcinogenic chemical compounds.

Epithelial Tissue and Types of Treatment—State of the Art

It should also be noted that the epithelial tissue does not have its own blood supply, which hinders or prevents the use of drugs or medications that depend on transportation routes, such as the circulatory or lymphatic system, for example, to reach body tissues of this particular region. This is relevant for the treatment of malignant lesions located in the most superficial layer of the lining epithelium, once the use of therapies containing agents of systemic action is not very effective, or is virtually useless for the treatment of malignant or pre-malignant lesions in the superficial layer of the epithelial tissue lining surfaces outside or inside body organs. Therefore, in the closest state of the art, the medical treatments used include special techniques and procedures, such as surgical procedures, radiotherapy and drugs with local or topical effect, which work more effectively in the epithelial tissue lining surfaces outside or inside the body.

The main goal of the topical treatment of tumors in this specific region is to eradicate tumors of the epithelial tissue lining surfaces outside or inside body organs before they become invasive, spread to adjacent tissues, and then enter the body. For the purposes of the present invention and to facilitate understanding of the state of the art it is important to mention that lining epithelial tissue can be accessed for therapeutic purposes, that is, for performing local excisions and topical treatments inside or outside the body.

This occurs, for example, in the case of skin tumors locally treated by surgical removal, radiation therapy, phototherapy and chemotherapy, which may be used alone or in combination. Likewise, epithelial tumors of the inner surface of an organ (e.g. urinary bladder) can be reached and topically treated by means of surgical procedures, various types of radiations and also drugs with topical action, after deposition in the regions of interest.

In the particular case of medicines, some special techniques and equipment can be used in the region of interest long enough to exert topical action in the affected tissue.

As an example, the treatment of tumors of the inner surface of the urinary bladder epithelium, treated by irrigation or contact with chemotherapy drugs, which for these purposes are introduced into the bladder with the use of catheters for insertion into the bladder. Finally, it is well-known in the closest state of the art that there are other types of non-drug therapies available to treat tumors that affect the lining epithelial tissue, e.g., surgical treatments and radiation therapy, which can also be used in combination or association with drugs to maximize the therapeutic response.

Safety Requirements for the Development of Therapies for Use in the Epithelial System

Medications or procedures to be used for the treatment of malignant lesions in the external or internal surfaces of the epithelial tissue should have topical action, restricted to the epithelium, to prevent them from reaching blood vessels and other transport systems of the body, causing toxicity to other organs.

As a general rule, it can be said that any procedure or medication developed for the topical treatment of malignant lesions of the epithelial tissue covering internal surfaces of the body can be used for the treatment of malignant lesions and tumors located in the epithelial tissue lining surfaces outside body organs. The opposite is not always possible, that is, some drugs and therapies developed for use in epithelial cells that line external surfaces of the body are not suitable for use in tumors located in the epithelium of internal surfaces because of high toxicity problems.

The reason for this is that, besides being more sensitive to aggressive agents, the epithelium that covers internal surfaces of the body is more sensitive to aggressive agents, and sometimes has a higher degree of permeability, compared to the epithelium that covers external body surfaces.

Differences in the degree of permeability between the epithelium of the internal and external surfaces of the body are directly related to the function performed by the organ covered by these tissues.

This is observed, for example, in the case of the epithelial cells that line the intestine, which can carry substances into the body and therefore can more easily absorb substances than the skin, which performs functions related to protection against penetration of external agents into the body and of elimination of substances from the body, as in the case of sweat.

In practical terms, this prevents products with high systemic toxicity, which can be used for therapeutic purposes in the skin because of its relatively low absorption capacity, from being used to treat diseases in the intestinal region, due to the high absorption capacity of the lining epithelium of this anatomical region.

As a practical example of a procedure used for the two types of epithelial surface, we can cite the treatment of cancerous lesions of the stomach lining, through the use of pulses of high energy light (laser), a procedure that is also widely used to eradicate malignant lesions in the skin.

Treatments based on corrosive chemical agents, such as those used to remove warts and malignant or premalignant lesions in the outer layer of skin (epidermis) cannot be used in the treatment of lesions in the epithelial tissue of internal surfaces due to their potential for indiscriminate damage to healthy tissues, and if these agents are absorbed by the body they can cause serious or fatal intoxication.

In the state of the art, we can cite Podophylin, a compound derived from a plant of the species Podophyllum Emodi, used for the removal of superficial skin lesions, including warts usually caused by HPV and also malignant and premalignant lesions. However, it cannot be used in the epithelial tissue lining surfaces inside body organs due to its toxic properties and because it can be absorbed into the body.

The drug imiquimod (IUPAC: 1-(2-methylpropyl)-1H-imidazo[4,5-c]quinolin-4-amine, C₁₄H₁₆N₄), an immunomodulator or local immune response modifier, which has high toxicity to various cell structures, can be cited as another example.

Consequently, in clinical practice, this compound is only allowed for the topical treatment of premalignant lesions caused by HPV virus, such as condyloma acuminata, or for skin papilloma, or else for the treatment of basal cell carcinoma in the epithelium of the skin surface.

Due to this toxicity, imiquimod cannot be used in the treatment of malignant lesions in the epithelium of internal surfaces of the body, such as, premalignant or malignant lesions in the intestine, uterus, stomach and urinary bladder, because it can be absorbed by the mucosa of these organs.

Finally, it should be noted that the main purpose of the treatment of malignant or premalignant lesions in the lining epithelium of internal or external surfaces of the body is the elimination of such lesions before they propagate into deeper layers, which unlike the surface layers are very irrigated, favoring the dissemination of cancer to other organs and systems of the body. When the lesion extends beyond the surface layer of the lining epithelium and reaches the basal layer or further, the treatment with drugs with predominantly topical action is not effective, since many compounds are only effective or can be used locally due to toxicity or permeability problems.

Since the treatment of malignant lesions that extend beyond the surface layer of the lining epithelium is complicated because of the above mentioned reasons, other types of therapies should be used, or else the use of chemotherapy drugs with systemic effect, which are usually toxic to the most of patients.

For example, local (intravesical) immunotherapy with BCG vaccine used in the topical treatment for cancerous lesions in the epithelial lining of the urinary bladder is ineffective to treat infiltrating tumors, that is, malignant tumors extending beyond the surface layer and invading the basal or muscle layer of the bladder, as it will be detailed in the present report. Furthermore, in the state of art, for treatment of urinary bladder cancer, due its high toxicity and the possibility of systemic infection of the patients, BCG vaccine is only indicated for intravesical use.

Therefore, the topical therapy in the closest state of the art, which includes most current therapeutic agents cited here in the case of urinary bladder cancer that infiltrates to the muscle layer cited here, for example, is ineffective, and consequently radical surgery for removal of the bladder, a procedure called cystectomy, followed or not by radiotherapy and systemic chemotherapy, is required.

In the state of the art, cystectomy, or partial or total removal of the urinary bladder, is the procedure indicated for the treatment of invasive tumors, and even for superficial bladder tumors that do not respond to standard therapy, which consists in local surgical excision of the tumor followed by immunotherapy or topical chemotherapy, as will be explained in detail herein.

On the one hand, this surgical procedure (cystectomy) can prevent further spread of cancer to new areas, but on the other hand, it causes significant loss of quality of life for most patients. For reference: Clark, Peter E. et al—Radical cystectomy in the elderly—Comparison of clinical outcomes between younger and older patients. Cancer-Volume 104, Issue 1, pages 36-43, 1 Jul. 2005.

The Need for Improvements in the State of the Art

The above examples clearly show that, due to the considerable shortcomings of the available treatments and drugs, new types of treatment for cancer originated or located in the epithelial lining of internal and external surfaces of the body are needed in the state of the art, to replace or complement the current treatments, maximizing their efficiency.

It would be highly desirable that these new therapeutic modalities had no unwanted side effects commonly observed in the treatments available in the state of the art or reduced toxicity to end users.

The creative or inventive activity should therefore be aimed to the discovery of new therapeutic modalities, including new methods of treatment and compounds, used alone or in association, and which are comparatively more beneficial than the current treatments in terms of effectiveness and safety.

If these therapeutic modalities, because of their characteristics or properties, can be used in the topical treatment of malignant tumors in the lining epithelium of internal and external surfaces of the body, without toxicity and contributing to reduce the toxicity of the current treatments, this would undoubtedly constitute a considerable improvement in the state of the art which can be used alone, or in combination or association with other therapies

The present invention satisfies all these needs, by providing a method of treatment using an immunomodulator, that can be used alone, or in combination or association with other therapies which, in an innovative way, allows the treatment of malignant tumors located in the epithelial lining of internal and external surfaces of the body, and does not cause significant toxicity that disallows its use, unlike most compounds available in the state of the art. And in order to demonstrate the usefulness, inventive level of activity involved and comparative advantages of the invention reported here, several practical examples of its use in malignant lesions in the epithelial lining of internal and external surfaces of the body in experimental animals are presented.

The results are presented only for illustrative purposes to help understand the novelty, usefulness and advantages of this invention, and by no means intend to limit the scope or the field of application of the present invention.

Cancer in the Epithelial Tissue Lining Surfaces Inside Body Organs—Cancer of the Urinary Bladder—State of the Art

Urinary bladder cancer is one of the most important cancers that affect or originate in the epithelial lining of inside surfaces. Bladder cancer is the most common tumor of the urinary tract after prostate cancer, and one of the most common cancers, with a male to female ratio of 4:1 in the United States. (American Cancer Society. Cancer Facts and Figures 2009. Atlanta, Ga. American Cancer Society; 2016).

From an economic point of view, patients with bladder cancer represent the highest annual costs for the treatment of all types of cancer in the United States, according to reports from the health sector for this specific disease (Simons M P, O'Donnell M A, Griffith T S. Role of neutrophils in BCG immunotherapy for bladder cancer. Urol Oncol 2008; 26:341-5, PubMed: 18593617) (James A C et al 2013).

Tumor Standardized Classification System—State of the Art—the TNM System (TNM Staging System)

The TNM system is one of the most widely used cancer-staging systems. This system has been accepted by the Union for International Cancer Control (UICC) and the American Joint Committee on Cancer (AJCC).

TNM System—Classification of Urinary Bladder Cancers

According to the TNM system, tumors in the urinary bladder can be classified as follows (Table 1).

TABLE 1
Classification of urinary bladder cancers using the TMN system

| | |
|---|---|
| TX | Primary tumor cannot be assessed |
| T0 | No evidence of primary tumor |
| Ta | Noninvasive papillary carcinoma |
| Tis | Carcinoma in situ: “flat tumor” |
| T1 | Tumor invades subepithelial connective tissue |
| T2 | Tumor invades muscle |
| | T2a - Tumor invades superficial muscle (inner half) |
| | T2b - Tumor invades deep muscle (outer half) |
| T3 | Tumor invades perivesical tissue |
| | T3a - microscopically |
| | T3b - macroscopically (extra vesical mass) |
| T4 | Tumor invades any of the following structures: |
| | prostate, uterus, vagina, pelvic wall or abdominal wall. |
| | T4a - Tumor invades prostate, uterus or vagina. |
| | T4b - Tumor invades pelvic wall or abdominal wall |

Approximately 75% of tumors located in the epithelial lining of the urinary bladder detected at the time of diagnosis are classified as non-muscle invasive bladder cancers, or else classified according to the TNM system as Ta, Tis, T1.

[symbol]

These tumors classified as Ta, T1, Tis, are confined to the most superficial mucosa of the urinary bladder or in the lamina propria. The remaining 25% of all the types, at the time of diagnosis, are classified as muscle invasive cancers, or else, into the groups T2, T3, T4 of the TNM System (Table 1). For reference: (Sullivan P S et al. Urine cytology and adjunct markers for detection and surveillance of bladder cancer. Am J Transl Res 2010; 2(4):412-440 and Babjuk et al—EAU Guidelines on Non-Muscle-Invasive Urothelial Carcinoma of the Bladder—Eur Urol 2008 August; 54(2): 303-14.

Treatment Strategies

For the purpose of preparation of therapeutic strategies, tumors are usually divided into: superficial and invasive, with superficial tumors including those classified into stages Tis, Ta and T1 by the TNM system (Table 1). According to this system, penetration of muscle layer by the tumor mass identifies invasive bladder tumors, that is, T2, T3 and T4 (Table 1).

Treatment

The initial approach for all bladder tumors is a surgical procedure called Transurethral Resection of Bladder Tumors or TURBT. The so-called Transurethral Resection of Bladder Tumors (TURBT) is the standard procedure for diagnosis, staging and initial treatment of tumors located in the epithelial lining of the urinary bladder. Briefly, transurethral resection of bladder tumors or TURBT comprises the surgical removal of tumors or lesions affecting the urinary bladder suspected to be malignant. Such tumors or lesions are removed (resected) with the aid of surgical handles, laser scalpels and/or electrocauterization

The surgical technique called Transurethral Resection of Bladder Tumors (TURBT) is well-known in the state of the art, and more detailed information can be obtained e.g. in EAU guidelines on the treatment of urothelial carcinoma, a non-muscle invasive bladder cancer. For reference: Babjuk et al—EAU Guidelines on Non-Muscle-Invasive Urothelial Carcinoma of the Bladder—Eur Urol 2008 August; 54(2): 303-14.

Subsequently, and at the end of the procedure, tissue fragments are removed from the region by aspiration, for conducting anatomopathological examination and histological assessment of the lesions found, for purposes of classification of the tumor, usually according to the TNM system, aiming to establish the need for and/or type of treatment patient will require.

As already mentioned, after completion of the TURBT, the tissue fragments removed are examined by several techniques, such as histological analysis of tissues to allow tumor classification using the TNM system, to establish the need for and/or type of treatment the patient will subsequently require.

As shall be explained in more detail in this report, for epithelial tumors classified as high grade by the TNM system (Ta, T1, Tis), the best procedure available in the state of the art is a local excision of the tumor (TURBT) for removal and staging of tumors, followed by adjuvant intravesical therapy using chemotherapy or immunotherapy compounds.

In the state of the art, the best method of treatment for tumors originated in or affecting the epithelial tissue lining surfaces inside or outside the body uses a combination of treatments involving the removal of lesions located by surgical procedures, followed by adjuvant therapy using drugs or compounds of topical action. For reference: Babjuk et al—EAU Guidelines on Non-Muscle-Invasive Urothelial Carcinoma of the Bladder—Eur Urol 2008 August; 54(2): 303-14. The main purposes of this postsurgical treatment in the case of tumors affecting the epithelial tissue lining inside or outside body surfaces, and in the particular case of bladder cancer, is the elimination of tumors that might have gone undetected during the surgical stage, and also prevent tumor recurrence.

Treatments Available in the State of the Art

Although any malignant tumor, including those in the epithelial region, can spread throughout the body, in the specific case of patients with tumor in the superficial epithelium of the urinary bladder, those classified as high degree Ta, T1, Tis (Table 1) have the highest risk of recurrence and progression, or else, of extending beyond the epithelium, becoming invasive and spreading throughout the body, if treated by transurethral resection only, or else, by local excision. Therefore, after surgical removal of the lesions (TURBT) in the epithelial lining of the internal surface of the organ, the best treatment available in the state of the art, to avoid progression or tumor recurrence is the treatment of the urinary bladder epithelium using chemotherapy and immunotherapy compounds.

In the specific case of urinary bladder cancer, the topical treatment of the vesical epithelium using chemotherapy or immunotherapy compounds is called intravesical therapy or adjuvant intravesical therapy.

The so-called intravesical therapy is a modality of topical treatment that, in the specific case of the epithelium or epithelial surface of the bladder, consists in the introduction of agents with anticancer properties in the bladder, through appropriate means, where they will remain for a certain period in contact with the affected epithelium.

The main purposes of the topical intravesical treatment are to minimize the possibility of persistence and tumor relapse, inhibit the formation of new tumors and ultimately attempt to avoid or slow the progression of superficial lesions located in the epithelium for other tissues or organs. For illustrative purposes only, some examples of therapeutic agents in the state of the art used in the treatment of malignant lesions in the epithelium of the urinary bladder are shown in Table 2.

According to their mode of action, these agents can be generally classified in two groups or classes: chemotherapy and immunotherapy drugs. The chemotherapy drugs comprise all compounds capable of destructive action against tumor cells, while those classified as immunotherapy drugs comprise compounds that act indirectly via the patient's immune system, which, in turn, stimulated by these drugs, is able to eliminate or slow the progress of the cancer.

TABLE 2
CHEMOTHERAPY AND IMMUNOTHERAPY
DRUGS MOST USED FOR INTRAVESICAL
APPLICATION IN BLADDER CANCER

| | POSSIBLE MECHANISMS OF ACTION |
|---|---|
| CHEMOTHERAPY | |
| DRUGS | |
| Mitomycin C | Antibiotic, inhibition of DNA synthesis. |
| Thiotepa | Alkylating agent |
| Doxorubicin | Inhibition of topoisomerase, inhibition |
| | of DNA synthesis. |
| Gemcitabine | Inhibition of DNA and RNA synthesis. |
| Valrubicin | Inhibition of topoisomerase, inhibition |
| | of DNA synthesis. |
| IMMUNOTHERAPY | |
| DRUGS | |
| Bacillus Calmette-Guérin | Stimulation of the immune system |
| (BCG vaccine) | |
| Interferon (IFN) | Stimulation of the immune system, |
| | lymphocyte activation, |
| | antiangiogenic, antiproliferative. |
| Interleukin-2 (IL-2) | Stimulation of the immune system |

Chemotherapy and Immunotherapy in Bladder Cancer—Main Therapeutic Agents

As previously mentioned, the treatment of malignant lesions in the lining epithelium involves the combination of different therapies, beginning with a surgery to remove the tumors followed by treatment of the affected region with the use of chemotherapy and/or immunotherapy to eradicate residual tumors and prevent disease recurrence.

In the closest state of the art, in the case of postsurgical treatment of malignant lesions in the epithelium of the urinary bladder, intravesical immunotherapy using agents capable of stimulating the immune system is considered the most efficient therapeutic procedure compared to the use of chemotherapeutic compounds, regarding the prevention of cancer recurrence. For reference: Hall M C, Chang S S, Dalbagni G, et al. Guideline for the management of nonmuscle invasive bladder cancer (stages Ta, T1, and Tis): 2007 update. J Urol. 2007; 178(6):2314-2330.

Among the agents that may be used topically for the treatment of epithelial cancer of the urinary bladder, the most effective immunotherapeutic agent used in the closest state of art is BCG vaccine, which was originally developed for the prophylactic treatment of tuberculosis. Alexandroff A B, Jackson A M, O'Donnell M A, James K. BCG immunotherapy of bladder cancer: 20 years on. The Lancet, Volume 353, Issue 9165, Pages 1689-1694, 15 May 1999.

The use of BCG vaccine in the postsurgical treatment of cancerous lesions in the epithelial lining of the urinary bladder began in the 70s with the pioneering work of Morales and collaborators, who developed a new and innovative practical use for a product that was previously used only for prophylaxis of tuberculosis. Alexandroff A B, Jackson A M, O'Donnell M A, James K. BCG immunotherapy of bladder cancer: 20 years on. The Lancet, Volume 353, Issue 9165, Pages 1689-1694, 15 May 1999.

Since then, it has become the leading postsurgical treatment used in the state of the art as adjuvant therapy in the treatment of epithelial cancer, especially epithelial tumor of the urinary bladder. For reference: Alexandroff A B, Jackson A M, O'Donnell M A, James K. BCG immunotherapy of bladder cancer: 20 years on. The Lancet, Volume 353, Issue 9165, Pages 1689-1694, 15 May 1999.

Classification of the Degree of Risk of the Lesions and Intravesical Adjuvant Therapy

Although the treatment of any epithelial lesion in the urinary bladder considered cancerous or with the potential to develop into cancer begins with the surgical removal of the tumor (TURBT), the subsequent treatment may vary, depending on the degree of classification of the cancer.

For tumors classified as having high degree of recurrence, though low risk of progression, the most commonly used criteria for treatment in the state of the art indicate the need for intravesical adjuvant therapy following surgical resection of lesions (TURBT), using intravesical agents.

Therefore, the recommendations of the AUA (American urological association) include the use of adjuvant therapy with chemotherapeutic agents, such as Mitomycin C or immunotherapeutic agents, such as BCG vaccine. For reference: Hall M C, Chang S S, Dalbagni G, et al. Guideline for the management of nonmuscle invasive bladder cancer (stages Ta, T1, and Tis): 2007 update. J Urol. 2007; 178(6):2314-2330

For patients with tumors classified as having high risk of recurrence and also high risk of progression (Ta, T1, Tis), the most common recommendation in the state of the art after surgical removal of the tumors includes an initial phase of therapy where only BCG vaccine induction is used followed by BCG maintenance therapy. For reference, see Hall M C, Chang S S, Dalbagni G, et al. Guideline for the management of nonmuscle invasive bladder cancer (stages Ta, T1, and Tis): 2007 update. J Urol. 2007; 178(6):2314-2330.

In the state of the art, according to the results obtained in several clinical trials, the use of intravesical therapy with BCG vaccine indicated that this immunotherapeutic drug when compared with chemotherapeutic agents such as Mitomycin C, was clearly more effective. For reference: Sylvester R J et al—Bacillus Calmette—Guérin versus Chemotherapy for the intravesical treatment of patients with carcinoma in situ of the bladder: A meta-analysis of the published results of randomized clinical trials. J Urol. 2005 July; 174(1):86-91; discussion 91-2.

For example, the results published by the American Urological Association (AUA) show a recurrence rate of 34%, after 5 years, for patients treated with BCG induction therapy, followed by BCG maintenance therapy, compared to a recurrence rate of 64% for patients treated with chemotherapy drugs (Mitomycin C). For reference: Sylvester R J et al—Bacillus Calmette—Guérin versus Chemotherapy for the intravesical treatment of patients with carcinoma in situ of the bladder: A meta-analysis of the published results of randomized clinical trials. J Urol. 2005 July; 174(1):86-91; discussion 91-2.

Intravesical Maintenance Immunotherapy

Maintenance therapy is defined in the state of the art as the periodical exposure of the epithelial tissue (urothelium) for considerable periods to intravesical antineoplastic agents such as Mitomycin C or BCG. The use of maintenance therapy has proved to be an important strategy for reducing cancer recurrence, as well as disease progression to more advanced and aggressive stages.

The use of BCG maintenance therapy, after transurethral resection of bladder tumors (TURBT), is considered the most effective treatment, in the state of the art, in reducing the rate of tumor recurrence compared to any other intravesical agents, according the results arising from the SWOG 8507 study, that is considered the largest clinical trial performed in US using BCG induction plus maintenance for treatment of patients with bladder cancer. For reference: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

The maintenance treatment in the SWOG 8507 lasted for 3 years. All patients were followed for 5 years after completion of their treatments for assessment of disease recurrence. However, only 16% of the patients in the SWOG 8507 study completed the entire period of intended use of the treatment due to problems related to toxicity of BCG. For reference: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

However, the rate of recurrence after 5 years was 60% for the patients treated with BCG maintenance therapy, compared to 41% of non-recurrence, in the same period of time, for the patients not treated with the referred therapy.

For references and results of the SWOG 8507 study: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

After the completion of this study, the use of BCG vaccine following transurethral resection of bladder tumors (TURBT), has definitely established itself in the state of the art as the best treatment option for malignant tumors at high risk of progression affecting the lining epithelium of the urinary bladder.

Complications and Main Adverse Effects of Intravesical Therapy With Drugs

Table 3 relates the most common adverse side effects associated with the use of several compounds used in intravesical therapy. The symptoms and effects related to the use of BCG are the most frequently cited in the literature available in the state of the art, reflecting the fact that it is the most frequently used adjuvant therapy (Table 3).

TABLE 3
Adverse side effects and complications of the drugs
used in the intravesical therapy for bladder cancer

| | | (%) patients |
|---|---|---|
| Drug | Adverse side effects | affected |
| Bacillus Calmette-Guérin | Cystitis | >50 |
| (BCG) | Fever (low grade) | 25 |
| | Prostatitis | <5 |
| | Epididymitis | <2 |
| | Fever (high grade) | 3 |
| | Septicemia | <0.5- |
| | Generalized infection | Rarely |
| Mitomycin C | Hematuria | 10 |
| | Myelosuppression | <10 |
| | Skin rash | <10 |
| | Bladder contraction | <2 |
| | Tissue necrosis | Rarely |
| Thiotepa | Chemical cystitis | 20 |
| | Myelosuppression | 10 |
| Doxorubicin | Chemical cystitis | 25 |
| | Allergic reaction | 1 |

Bibliographic references (Table 3): Hall M C, Chang S S, Dalbagni G, et al. Guideline for the management of nonmuscle invasive bladder cancer (stages Ta, T1, and Tis): 2007 update. J Urol. 2007; 178(6):2314-2330

Adjuvant Immunotherapy With BCG—Practical Problems and Contraindications

Although the BCG vaccine used as therapeutic adjuvant in the topical treatment of bladder cancer is the most effective therapy available in the state of the art to prevent tumor recurrence and progression after transurethral resection of bladder cancer, a large number of patients experience recurrence and progression of the disease, despite undergoing the referred treatment. The treatment with BCG therapy is ineffective in around 30 to 40% of the patients, and even among those patients who respond well to treatment in the early days, 30% will suffer a relapse, that is, recurrence of bladder epithelium cancer after a certain period of use of BCG therapy. For reference, we cite: Palou Redorta J. Management of BCG Failures, Eur Urol. (2006; 49:779-80)

Additionally, the application of BCG vaccine in intravesical treatment of bladder cancer is considerably hindered in practice by the occurrence of unwanted side effects, which are detected in around 90% of the patients, consisting of symptoms of irritation, which lead to presence of blood in urine (haematuria), dysuria, causing early discontinuation of treatment in many cases. For reference—Askeland et al. Bladder Cancer Immunotherapy: BCG and Beyond. Advances in Urology. Volume 2012 (2012), Article ID 181987. http://dx.doi.org/10.1155/2012/181987.

In a clinical study conducted in 2000, in the United States, by the Southwest Oncology Group (SWOG 85071), only 16% of the total number of patients who received intravesical BCG to treat bladder cancer completed the treatment because of unwanted side effects. For reference: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

Additionally, because of episodes of infection, including systemic infection by a microorganism (Mycobacterium bovis) used in the preparation of BCG vaccine, doses need to be reduced, prolonged and expensive treatment with antibiotics are required and finally in some cases, interruption of BCG therapy. For reference: Lamm D L, Blumenstein B A, Crissman J D, et al. Maintenance bacillus Calmette-Guérin immunotherapy for recurrent TA, T1 and carcinoma in situ transitional cell carcinoma of the bladder: a randomized Southwest Oncology Group Study. J Urol 2000; 163:1124-9. See also: Palou Redorta J. e Management of BCG “Failures”. Eur Urol. (2006; 49:779-80).

Finally, according to recommendations of the American Urolological Association (AUA), the BCG vaccine is not recommended for use in intravesical treatment of tumors in patients under the following conditions: a) in the two weeks following bladder surgery (TURBT), b) shortly after traumatic bladder catheterization, c) in the presence of hematuria, d) in pregnant or breast feeding patients, e) in immunosuppressed patients or in patients taking drugs with immunosuppressive effects, f) in patients under radiotherapy, g) in patients with urinary tract infection and with a history of allergic reactions or who had previous reactions to BCG. For reference: Hall M C et al. Guideline for the Management of Nonmuscle Invasive Bladder Cancer: (Stages Ta, T1, and Tis): Update (2007). J Urol. 2007 December; 178(6):2314-30.

Non-Surgical Options for the Case of Failure of Immunotherapy With BCG—State of the Art

As set forth, in the closest state of the art, the best therapy available to treat urinary bladder cancer, which is BCG vaccine, has important limitations because of the high rate of cancer recurrence and the high incidence of unwanted side effects, leading patients to discontinue treatment.

BCG toxicity leads to early interruption of treatments for most of patients, a condition also known as BCG intolerance, that emerges in the setting of treatment of bladder cancer, that in turn favor the recurrence of cancer lesions as well as progression of disease to deadly invasive and metastatic presentations.

Clearly, the condition named BCG intolerance is related to insufficient amounts of BCG received, as the treatment was interrupted due to BCG toxicity (van der Meijden A P et al 2003, Takeda T et al 2009).

Tough BCG reduces the recurrence and progression risk in NMIBC patients, the outcomes are only observed when maintenance therapy is carried out (Lamm D et al, 2000, Lamm et al, 2010, Brausi M et al 2014). Evidences from studies in animal models provides further confirmation that the protective immunity induced by BCG wanes with time, providing the rationale for the use of maintenance (Lamm D et al 2010).Thereby, the risk of disease recurrence increases for patients who interrupt their BCG treatments due its local or systemic toxicity (Takeda T et al 2009).

According clinical studies enrolling a large number of patients, the most episodes of local and systemic toxicity of BCG emerge during induction phase or in the first half year of maintenance therapy. The main cause of delays and interruptions of BCG instillations is its local toxicity, such as localized inflammation leading to cystitis and macroscopic haematuria, which affect up to 75% of patients, leading to a withdrawal from BCG treatment in around 15% of patients (van der Meijden A P et al 2003, Brausi M et al, 2014).

Tough less frequent, systemic complications, such as fever, affects around 15% of patients leading to withdrawal from treatment in around 5% of them. The consequences of interruption of treatment are deleterious, as the effects of BCG against the disease recurrence and progression are only achieved when maintenance therapy is carried out (van der Meijden A P et al 2003, Brausi M et al, 2014).

Of note, BCG toxicity can lead to a low adherence to guidelines among urologists, including underutilization of intravesical therapy, even in the treatment of high-grade NMIBC patients (Witjes J A et al 2013). A study based on data from Nacional Cancer Institute Surveillance, Epidemiology, and End Results Program (NCI/SEER) indicated that BCG was used in less than one quarter of eligible patients, despite the clinical guidelines recommending its use (Spencer B A et al 2012). As the drug has caused more side effects than intravesical chemotherapy, urologists are still not confident to use BCG, mainly in maintenance schedules, despite reductions of tumor recurrence and progression observed in the maintenance therapy.

In the case of failure of BCG vaccine therapy, which includes both tumor recurrence and discontinuation of treatment and even abandonment of treatment after episode of severe toxicity for the user, without tumor removal, the most indicated treatment in the state of the art is total or partial removal of the urinary bladder, which is called cystectomy.

Cystectomy is a high-risk surgery that invariably results in significant worsening of the quality of life of patients, and, thus, it should be avoided or delayed as much as possible. In the closest state of the art, one of the possible options of treatment for the case of failure of intravesical therapy with BCG, aiming to avoid or delay total bladder removal (cystectomy) is its replacement in the intravesical treatment of the lining epithelium of the urinary bladder by less effective chemotherapeutic agents such as Mitomycin C, Thiotepa, Adriamycin, Valrubicin, Gemcitabine and some others.

However, such alternative chemotherapeutic agents, (e.g. doxorubicin, thiotepa, mitomycin, gemcitabine) have been used in the setting of BCG-refractory disease but have limited activity and are considered investigational in this setting]. Thus, the recommended standard of care for patients with bladder cancer who have failed after intravesical BCG treatment has been to proceed with cystectomy.

Although the use of agents such as chemotherapy drugs can be considered a valid option for treating epithelial tumors of the urinary bladder classified as low grade, this does not apply to patients with high-grade cancer, i.e., classified according to the TNM systems in grades Ta, T1, Tis.

For example, in a study conducted in 1999, only 19% of patients with bladder cancer in use of intravesical chemotherapy (Mitomycin C), had no recurrence of cancer during the follow-up period, compared with 39% of the patients in use of BCG that are disease free. For reference: (Malmstrom P U, Wijkstrom H, Lundholm C, et al. 5-year follow-up of a randomized prospective study comparing mitomycin C and bacillus Calmette-Guérin in patients with superficial bladder carcinoma. J Urol 1999; 161:1124-7.

To resume, the use of intravesical chemotherapy in urinary bladder cancer with the main compounds available in the state of the art, after failure in BCG therapy, either by abandonment of treatment with BCG vaccine, or because of their high toxicity, remains highly experimental and so far did not achieve the same degree of success obtained with the use of BCG intravesical therapy. Without any doubt, improvements in the success rate of such alternative treatments will be welcome.

Representing a clear advance over the prior art in the treatment of bladder cancer, the claimed invention provides a new method to maximizes synergistically the efficacy of these alternative intravesical treatments to be used in case of BCG or other drugs failure, as proved by several practical examples of its use provided in the Specification.

The Need for Improvements in the State of Art Concerning Treatment of Blader Cancer

The failures in BCG therapy, which is the best treatment available in the closest state of the art for urinary bladder cancer, either by its high toxicity that leads to premature abandonment, or by its relative ineffectiveness evidenced by the high rate of cancer recurrence, even for patients who completed the full treatment, clearly indicate the urgent need for improvements in the state of the art, through the provision of new treatments and drugs.

Surgical Treatments for Bladder Cancer (Cystectomy)

As previously mentioned in the closest state of the art, the main therapy indicated for patients who do not respond to the best therapy available in the state of the art for bladder cancer, i.e., BCG intravesical therapy or chemotherapy following transurethral resection (TURBT), is partial or total removal of the urinary bladder due to the high risk of progression of cancer lesions to more invasive and/or metastatic forms.

This procedure is also indicated for patients who had invasive tumors (T2, T3, T4-TNM System) at the time of diagnosis. The total or partial removal of the urinary bladder, called cystectomy, is a high-risk surgical procedure and that usually involves a high degree of morbidity and mortality. In a study with 1054 patients who underwent radical cystectomy due to bladder cancer, almost all had postsurgical complications of various degrees, including serious complications and death associated with this type of surgery. For reference and an example of the state of the art: Clark, Peter E. et al—Radical cystectomy in the elderly—Comparison of clinical outcomes between younger and older patients. Cancer-Volume 104, Issue 1, pages 36-43, 1 Jul. 2005.

In addition, cystectomy strongly negatively affects multiple aspects of quality of life, with deleterious consequences including urinary incontinence, loss of sexual function, low self-esteem, and reduced social interaction. Finally, patients who refuse or who do not qualify for bladder removal face an increased risk of progression to muscle-invasive disease.

Alternative Treatments for Patients Who Cannot Undergo Surgery (Salvage Therapies)

Due to the high probability of occurrence of morbidity or postsurgical complications, as above explained, cystectomy is considered a high-risk procedure, and, thus, a large number of patients cannot undergo or refuse this type of surgery because of other pre-existing medical conditions and/or age-related conditions, such as heart problems, diabetes and others.

For patients who do not respond to standard therapy, i.e. surgical removal of lesions followed by intravesical chemotherapy (e.g., Adriamycin, Valrubicin) or immunotherapy (BCG, Interferon, IL-12), either due to medication intolerance (BCG), or failure to respond to any of the available therapies, and who have cancer recurrence, and additionally cannot undergo cystectomy, the existing alternatives are restricted to support measures, palliative measures using chemotherapy drugs (e.g. Valrubicin) and radiotherapy, to try to prolong patients' life for as long as possible-For reference: Toni K Choueiri and Derek Raghavan-Chemotherapy for muscle-invasive bladder cancer treated with definitive radiotherapy: persisting uncertainties-Nature Clinical Practice Oncology (2008) 5, 444-454.

In the United States, a chemotherapy drug called Valrubicin (N-trifluoroacetyladriamycin-14-valerate) is topically used (intravesical therapy) for palliative treatment of patients with urinary bladder cancer who cannot undergo surgery (cystectomy).

Despite its low efficacy, as results are seen in only 20% of the patients treated, this specific chemotherapy drug (Valrubicin) is currently the only chemotherapeutic agent approved by the FDA for the palliative treatment of patients with bladder cancer resistant to topical treatment with BCG and who cannot undergo surgery for removal of the urinary bladder (cystectomy) because of comorbidities such as concomitant disease, advanced age and intolerance to anesthetic procedures. For reference: Dinney C P et, Greenberg R E, Steinberg G D. Intravesical valrubicin in patients with bladder carcinoma in situ and contraindication to or failure after bacillus Calmette-Guérin. Urol Oncol. 2013 November; 31(8):1635-42. doi: 10.1016/j.urolonc.2012.04.010. Epub 2012 May 9.

Need for New Products and Therapies

Considering the evident shortcomings of the existing treatments in the closest state of the art for urinary bladder cancer, either surgical procedures (total or partial cystectomy, cryotherapy, laser), radiotherapy or drug treatments using chemotherapeutic compounds (ex: Thiotepa, Mitomycin C, Valrubicin) or immunotherapeutic compounds (BCG, exogenous interferons, IL-2, IL-12), there is urgent need in the state of the art for new modalities of treatment of epithelial cancer, including urinary bladder cancer, that could replace or complement the existing treatments, maximizing their efficiency.

It would be highly desirable that these new treatment modalities had no unwanted side effects commonly observed in the treatments available in the state of the art, as described in the present report.

The creative or inventive activity should therefore be aimed to the discovery of new therapeutic modalities, including new methods and products, used alone or associated, and which are comparatively more beneficial than the current treatments in terms of effectiveness and safety, with lower toxicity to the end user.

The present invention, in an innovative way, provides a new method of treatment of tumors of the superficial epithelium, including those located in the epithelium of the urinary bladder, through the use of an immunomodulator (proteic aggregate of ammonium and magnesium phospholinoleate-palmitoleate anhydride), which proved to be able to be used topically, alone, or in combination or association with the existing treatments, replacing them or reinforcing their effectiveness, and at the same time without the occurrence of unwanted side effects, which represents a significant advancement over existing treatments in the closest state of the art, as will be detailed in this report, with examples of practical use.

On the contrary, as will be shown in this report, the use of the referred substance, an immunomodulator, combined to the drugs and non-drug therapies in the closest state of the art, led to a noticeable decrease in the frequency of the unwanted side effects of these other treatments.

The present invention was derived from compound already known in the state of the art and described in (PI-0305373-3, US 2006/0093628 A1, EP 1529784 A1, WO 2011082458A1), though used in a way that is entirely different from the previous forms of administration and use described in the state of the art, with outstanding and unprecedented practical results, as shall be detailed by means of descriptions and experiments in this report.

The Invention

It is widely known in the state of the art that the best available strategy for treating tumors in the epithelial tissue lining surfaces outside and inside the body, especially tumors of the urinary bladder, is based on surgical removal of the tumor or affected tissue followed by adjuvant topical treatment with drugs, in order to avoid cancer recurrence or else to slow down its progression when complete elimination is not possible. The use of compounds or drugs in the intravesical treatment of tumors located in the epithelial tissue lining surfaces outside and inside the body for topical application, such as urinary bladder tumors, is facilitated by the relatively unobstructed access to these anatomical regions, such as the skin and hollow organs as the urinary bladder.

This anatomical peculiarity allows access for topical application of compounds, by means of several techniques or special equipment, such as catheters. In view of this peculiarity, any person skilled in the art can easily understand the advantages of topical treatment with drugs that act specifically on this type of tissue. Finally, the topical use of drugs aims to ensure that the maximum safe therapeutic drug concentration reaches the most superficial epithelial surface where the tumors are located.

Therefore, considering the possibility of access to such regions, and the limited or no blood supply to these regions, the use of compounds with topical action is essential to ensure new effective treatments of malignant or premalignant lesions in the epithelial tissue lining outside or inside surfaces.

For example, the treatment of tumors in the bladder's internal lining benefits from the fact that the anatomy of the body provides access from outside for the introduction of drugs or solutions, with the use of catheters that penetrate the organ through the urethra and, then, accessing the inside lining of the urinary bladder.

This type of application, that is, the topical action of drugs in organs with cavities or interior spaces, can be and is used for the treatment of cancers in the lining epithelium of other organs, besides the urinary bladder, such as the uterus, stomach or esophagus.

Given these peculiarities and the problems cited, and for the purposes of the invention, that is, providing a novel and effective method of treatment capable of topical action in the epithelial tissue lining outside and inside surfaces of body organs, a compound with the following properties and capabilities was required:

A) can be used for therapeutic effects against malignant or premalignant lesions located both in the epithelium lining outside and inside surfaces;

B) capable of producing therapeutic effect alone or combined to other drug and non-drug treatments;

C) presenting no significant toxicity;

D) capable of eliminating or minimizing toxic effects of other treatments;

E) capable of maximize and/or synergize the effects of others therapies eventually used in association or combination.

The invention, in an innovative and remarkable way, as shall be detailed in this report, with examples of its practical use, allows meeting all these requirements, that is, the above-mentioned items A, B, C, D and E.

The invention was developed based on new and surprising scientific data from the use of invention in human beings, and also experiments with animals involving comparative assessment of topical treatments for epithelial cancer, including urinary bladder cancer.

These experiments involved the comparison in animal models of several compounds already known in the state of the art, including BCG vaccine, chemotherapeutic drugs such as Mitomycin C, Gemcitabine, Docetaxel and Valrubicin, and also the proteic aggregate of ammonium and magnesium phospholinoleate-palmitoleate anhydride described in the state of the art in PI-0305373-3, US 2006/0093628 A1, EP 1529784 A1, WO 2009/097670A1, WO 2011082458 A1.

Analysis of data of these new experiments and scientific studies revealed effects and biological and therapeutic properties unprecedented in the state of the art for the immunomodulator (proteic aggregate of ammonium and magnesium phospholinoleate-palmitoleate anhydride). These new unprecedented properties in the state of the art were incorporated in the present invention.

In animal models and human subjects, when applied in epithelial tissues affected by cancer, the immunomodulator (proteic aggregate of ammonium and magnesium phospholinoleate-palmitoleate anhydride) in an outstanding and unprecedented way showed, in clinical practice, its effective topical action on the epithelial region affected by the cancer.

This unprecedented property of the referred substance (proteic aggregate of ammonium and magnesium phospholinoleate-palmitoleate anhydride), that is, its ability (until now unknown in the state of the art) to act against malignant and premalignant lesions when applied directly where its action is desired, has allowed its use in the present invention as topical medication for the treatment of malignant or premalignant lesions that affect the epithelial tissue lining outside or inside surfaces.

Additionally, the ability of the immunodulator (proteic aggregate of ammonium and magnesium phospholinoleate-palmitoleate anhydride) when used alone in the present invention for the topical treatment of malignant lesions, represents a major advance compared to the closest state of the art, since it proved to be much more effective than the main drugs treatments used in the state of the art, when compared to the latter in practical uses (Tables 4, 5, 6-A, 6-B, 6-C, 6D, 7, 8, 9, 10).

In a remarkable and unprecedented way, the immunomodulator (proteic aggregate of ammonium and magnesium phospholinoleate palmitoleate anhydride) not only maximized synergistically the effect of other drugs already used in the state of the art (Tables 4, 5, 6-A, 6-B, 6-C, 6-D,) when used in combination or association, and also when topically applied, but also significantly decreased the toxicity of treatment for the end user, in the association, with these remarkable and unprecedented practical advantages of its use being added for the purposes of the invention.

When a combination of drugs or treatments is used in clinical practice with a biological or therapeutic effect that is wider than the effects of its isolated components, this remarkable event is called synergy or synergistic effect of the referred combination or association. In the case of association of the immunomodulator (proteic aggregate of ammonium and magnesium phospholinoleate-palmitoleate anhydride) with other drugs, including BCG vaccine, Mitomycin C and Valrubicin, for instance, in the topical treatment of urinary bladder cancer, the synergy or synergistic effect provided by the invention consists in the maximization of the therapeutic effect of the association, using smaller dosages, as well as providing a remarkable decrease in toxicity for the association. (Tables 4, 5, 6-A).

This unprecedented ability of the immunomodulator (proteic aggregate of ammonium and magnesium phospholinoleate-palmitoleate anhydride) of acting topically on the epithelial tissue lining outside and inside surfaces, and without toxicity for the region treated, allows it to be used as topical treatment of malignant and premalignant lesions, alone or in association or combination with other therapies and procedures, with remarkable advantages compared to the treatments that were known until now in the state of the art.

Absolute Novelty of the Invention—Topical Use of the Immunomodulator Proteic Aggregate of Ammonium and Magnesium Phospholinoleate-palmitoleate Anhydride for the Treatment of Cancer in the Epithelial Tissue—State of the Art

Until now, it was reported in the state of the art (PI-0305373-3 US 2006/0093628 A1, EP 1529784 A1, WO 2011082458A1) that the immunomodulator (proteic aggregate of ammonium and magnesium phospholinoleate-palmitoleate anhydride) had antitumor activity only when it acted systemically, that is, by means of stimulation and/or modulation of the immune system, and this effect would only occur when the compound was introduced into the body by parenteral route, using subcutaneous, intramuscular or intraperitoneal injections.

Or, in other words, the immunomodulator (proteic aggregate of ammonium and magnesium phospholinoleate-palmitoleate anhydride) was always administered as an injectable drug for the treatment of cancer and other diseases, and once injected into the body would act systemically by stimulating the immune system.

In fact, in PI-0305373-3 US 2006/0093628 A1, EP 1529784 A1 and in other references in the state of the art (WO 2009/097670 A1), it is reported that the immunomodulator (proteic aggregate of ammonium and magnesium phospholinoleate-palmitoleate anhydride) has systemic therapeutic action, and, therefore, should be administered by injection (intramuscular, intraperitoneal and subcutaneous) in the body.

However, the best therapeutic strategy available in the closest state of the art for the treatment of urinary bladder cancer, as well as other tumors affecting the epithelial tissue lining outside or inside surfaces, also requires that topical treatment is adopted after surgical removal of the malignant or premalignant lesions, in order to prevent the recurrence of cancer.

This particular form of treatment that represents the closest state of the art for the treatment of tumors affecting the epithelial tissue of outside and inside surfaces of the body uses drugs with topical action based on the fact that compounds that act systemically and/or depend on internal transport routes, such as the circulatory or lymphatic system, are ineffective when used in the treatment of malignant or premalignant lesions in the epithelial tissue lining inside or outside surfaces of the body.

The explanation for this phenomenon that obstructs or neutralizes the benefits of drugs with systemic action is that the superficial regions of the internal and external epithelium have limited blood supply, which hinders or even prevents the access of an adequate concentration of drugs to the site of the malignant or premalignant lesions, when these medications depend on internal transportation routes, e.g. injectable drugs, as will be detailed in the present report.

As reported in the state of the art, the immunomodulator (proteic aggregate of ammonium and magnesium phospholinoleate-palmitoleate anhydride) was always ineffective when used in cancer cells in vitro, or in other words, this ability to be applied directly to the region where its action is desired was entirely unknown until now in the state of the art.

We cite US 2006/0093628 A1: “Cytotoxic properties. Cytotoxicity of the compound of the present invention was studied in the range of 10⁻⁴ to 10⁻⁸ M in 53 tumor cell cultures representing 8 distinct tumor types (lung, colon, central nervous system, melanoma, and ovary, kidney, prostate and mammary) and leukemia. The compound of the present invention did not display cytotoxicity for these tumor cells.”

Thereby the immunomodulator (proteic aggregate of ammonium and magnesium phospholinoleate-palmitoleate anhydride) in the state of the art had no topical action against cancer cells, once when directly applied to regions with several tumor strains (US 2006/0093628 A1, EP 1529784 A1) showed no toxicity, that is, was unable to damage or eliminate cancer cells when directly applied.

Therefore, the immunomodulator (proteic aggregate of ammonium and magnesium phospholinoleate-palmitoleate anhydride) has always been used as a systemic drug injected into the body, generating an effective immune response against cancer and also intracellular parasites. (US 2006/0093628 A1, EP 1529784 A1, WO2011082458 A1, WO 2009/097670 A1).

Nevertheless, when the immunomodulator (proteic aggregate of ammonium and magnesium phospholinoleate-palmitoleate anhydride) was used in experiments in animal models for the study of urinary bladder cancer, for the purpose of comparison with drugs with topical action, including chemotherapy and immunotherapy drugs (BCG vaccine). In these experiments, the immunomodulator was used in the injectable form and topically applied at the same concentrations. (Tables 4, 5, 6-A, 6-B)

In a remarkable and unprecedented way, contrary to all previous information in the state of the art, the substance was found to have potent antitumor action when introduced by a catheter in the epithelial tissue lining the urinary bladder of experimental animals (Table 4, Table 5, Table 6-A, Table 6-B, Table 6-C, Table 6-D, Table 9).

In one of the above cited experiments, the immunomodulator (proteic aggregate of ammonium and magnesium phospholinoleate-palmitoleate anhydride) was also experimentally used, in its injectable form, in the animal model for the study of bladder cancer, and showed no comparable results at the concentrations used (Table 4-Group E) when compared to the same compound, though topically applied. (Table 4-Group-D).

Now, in a remarkable way, the immunomodulator (proteic aggregate of ammonium and magnesium phospholinoleate-palmitoleate anhydride) that always was reported in the state of the art as being effective against infections and cancer when systemically administered by injection, has shown a surprisingly topical action against cancer in the inner lining (epithelium) of the bladder (Tables 4, 5, 6-A, 6-B, Table 6-C, Table 6-D, Table 9).

This unprecedented action, that is the topical action of the immunomodulator (proteic aggregate of ammonium and magnesium phospholinoleate-palmitoleate anhydride) against cancer affecting the epithelial tissue, when topically used, that is, without the need to be administered by injection, was incorporated for the purposes of the present invention. This unprecedented ability will be detailed in the present report by means of examples of its practical use in animals and also in trials with humans.

The immunomodulator (proteic aggregate of ammonium and magnesium phospholinoleate-palmitoleate anhydride), in the way it was used in the present invention, showed therapeutic properties when directly applied to the affected epithelial tissue, e.g. after being introduced in the bladder of experimental animals (Tables 4, 5, 6-A, 6-B, Table 6-C, Table 6-D, Table 9), through a catheter, added to saline suspension, poloxamers or else in cream formulation to be topically applied in humans (Table 7, table 8-B) and other formulations (Table 6-B), as described, also with examples of its practical use in the present report.

These results are totally unprecedented and remarkable, once all data from the scientific literature and other information available in the state of the art so far provided indications that the product (proteic aggregate of ammonium and magnesium phospholinoleate-palmitoleate anhydride) was only effective against cancer and other diseases when systemically administered, that is, indirectly acting by triggering a immune response of the body after being injected in animals and humans.

And also, when used for the treatment of urinary bladder cancer, the immunomodulator (proteic aggregate of ammonium and magnesium phospholinoleate-palmitoleate anhydride) was found to be more effective than the best topical treatment available in the state of the art for urinary bladder cancer, which is BCG vaccine, as will be demonstrated in the present report by means of examples of its practical use.

Finally, and also in a remarkable way, the immunomodulator (proteic aggregate of ammonium and magnesium phospholinoleate-palmitoleate anhydride) did not show adverse side effects in the inner mucosa (epithelium) of the organ, compared to the most advanced intravesical medication in the state of the art for the treatment of bladder cancer at high risk of progression (Ta, T1, Cis), that is, BCG vaccine (Tables 4, 5, Table 6-A). On contrary, while all animals with urinary bladder cancer treated with BCG vaccine and other drugs showed urinary bleeding (hematuria) during and of post-treatment, the animals treated with the immunomodulator (proteic aggregate of ammonium and magnesium phospholinoleate-palmitoleate anhydride) alone or in association showed no sign of urinary bleeding. In addition, microscopic examinations of the tissues of the animals revealed signs characteristic of cancer reversion. (Table 4, Table 5, Table 6-A, Table 6-B).

Reversion of the tumor process, that is, the curative effect, occurred when the immunomodulator (proteic aggregate of ammonium and magnesium phospholinoleate-palmitoleate anhydride) was used alone and also associated to other compounds in the treatment of urinary bladder cancer, maximizing the therapeutic effectiveness of the association, and even eliminating hematuria, which is demonstrated with an example of the practical use of the invention in animal model (Table 5 and Table 6-A).

In this particular aspect, or else, when associated to other drugs such as BCG, Mitomycin C, Gemcitabine, Docetaxel and Valrubicin, the use of the immunomodulator (proteic aggregate of ammonium and magnesium phospholinoleate-palmitoleate anhydride) not only proved to be able to maximize the effectiveness against cancer lesions in the inner epithelium (urinary bladder), but also, in a more remarkable way, created a synergistic effect for the association, since it remarkably maximizes the anticancer effects of the association of drugs and simultaneously decreased the toxicity associated to the treatment, which is demonstrated by the elimination of hematuria in the experimental animals (Table 5 and Table 6-A, Table 6-B, Table 6-C, Table 6-D).

The reversion of premalignant and malignant lesions in the epithelium lining internal and external surfaces (skin) in humans, is also sufficiently demonstrated in the present report, so that any expert with knowledge of the state of the art understands that the invention can be used without any obstacle in the treatment of malignant or premalignant lesions of this anatomical region, with the benefits of topical application, which is also one of the purposes of the present invention (Tables 7 and Tables 8-A and 8-B).

Purposes of the Invention

Therefore, one of the purposes of the present invention is to provide a compound for use in a method of topical treatment of malignant and premalignant lesions in the epithelial tissue lining surfaces inside and outside the body.

Another purpose of the present invention is to provide a compound for use in a method of topical treatment of tumors specifically located in the urinary bladder, which was found to be more effective than the main drugs and treatments in use in the state of the art for this type of disease.

Another purpose of the invention is to provide a method of treatment using an immunomodulator in association or combination with other types of treatments (drug or non-drug treatments) intended for treatment of malignant and premalignant lesions in the epithelial tissue lining inside and outside surfaces, providing synergistic effects without additional toxic effects.

Another purpose of the invention is to provide a method for maximization of antineoplastic effects of the current intravesical chemotherapy used in the state of art to replace BCG in the treatment of patients suffering of bladder cancer.

Another purpose of the invention is to provide a method for replacement of compounds used in the state of art for treatment of malignant and premalignant lesions in the epithelial tissue lining inside and outside surfaces, in case of emergence of toxic side effects or lack of efficacy of such therapies.

The present invention also aims to provide a new and powerful treatment able to maximize therapeutic effectiveness when used in combination or association other drugs in the state of the art intended for topical treatment of malignant and premalignant lesions in the epithelial tissue lining inside and outside surfaces of the body.

The present invention also aims to provide a new drug treatment without significant toxicity for users compared to drugs used in the state of the art intended for topical treatment of malignant lesions in the epithelial tissue lining inside and outside surfaces of the body.

Additionally, the present invention also aims to provide a new and powerful therapy able to create a synergistic therapeutic effect when combined to other drugs in the state of the art, intended for topical treatment of malignant lesions in the epithelial tissue lining inside and outside surfaces, due to its ability to maximize the therapeutic effectiveness of the combination or association, and simultaneously reduce the toxicity of the association for end users.

The present invention also aims to provide a new and powerful therapy able to replace the existing drug treatments, particularly BCG vaccine and other drugs used in the topical treatment of malignant lesions in the epithelial tissue lining inside surfaces, particularly in bladder cancer, in the event that a satisfactory result is not obtained with BCG vaccine, or else, such treatment needs to be replaced by other treatments due to unacceptable toxicity levels, which are intolerable to users.

The present invention aims to provide a new palliative treatment (salvage therapy) against malignant lesions in the epithelial tissue lining inside and outside surfaces to be used when, in the event that the medication available in the state of art fails, more aggressive treatments or that related to at higher risk of morbidity and mortality, such as surgical removal of the urinary bladder (cystectomy) or other forms of treatments that eliminate lesions, cannot be performed due to unacceptable risk to the patients.

The present invention is aimed to provide pharmaceutically acceptable carriers as herein described for use in the topical treatment of the lesions.

The present invention is aimed to provide topical formulations, using pharmaceutically acceptable carriers for use in treatment of the malignant or premalignant lesions in subjects in need thereof.

Finally, in order to sufficiently demonstrate the properties and benefits of the invention compared to the main products existing in the state of the art, several examples of its practical use are reported in this specification. These experiments of the practical use of the invention and its results are detailed in the specification to demonstrate the inventive activity, the absolute novelty and the uses and comparative benefits of the invention. These experiments are presented for illustrative purposes only, and by no means intend to limit the scope and the field of application of this invention.

Without the need for additional information, it can be affirmed that any person skilled in the art, based only on the information and examples of its practical use that will be provided in this report, should be able to understand and use the present invention to its fully extent.

Although the invention is intended for human use, it can be also used in the treatment of malignant or premalignant lesions in animals, which also have epithelial tissue that lines inside and outside surfaces of their bodies and can also be affected by tumors in these anatomical regions.

Practical Example of Use of the Invention in the Treatment of Bladder Cancer—Comparison With Bcg Vaccine—Experiment No.1—Table 4

Objective: Comparative assessment of the therapeutic response in animals subjected to experimental induction of bladder cancer with n-methyl-n-nitrosourea (MNU) Fifty animals (n=50) were used in the experiment.

Of these, 40 animals were given 1.5 mg/kg of n-methyl-n-nitrosourea (MNU) dissolved in 0.30 ml of sodium citrate, intravesically, with the use of a catheter, for 7 weeks. One group (control—GROUP-A) of 10 animals was only given saline solution (NaCl 0.9,%) also intravesically and for an equal period. The compound n-methyl-n-nitrosourea (MNU) was applied in the animals to experimentally induce immunosuppression and the subsequent production of urinary bladder carcinoma. After treatment with MNU, the animals were divided into 5 groups of 10 animals each.

After the use of MNU for 7 weeks, 4 batches of 10 animals each were subsequently subjected to intravesical instillation of 0.30 ml of 0.9% saline solution (GROUP-B-MNU), 10⁶ CFU (40 mg) of intravesical BCG (GROUP-C) and 10 mg/kg of the immunomodulator (proteic aggregate of ammonium and magnesium phospholinoleate-palmitoleate anhydride—GROUP-D), suspended in saline, once a week, for all the groups, using a catheter, during the entire treatment period of 14 weeks.

For the purposes of comparison, 10 mg/kg of the immunomodulator (proteic aggregate of ammonium and magnesium phospholinoleate-palmitoleate anhydride) was also used, however administered as a subcutaneous injection (GROUP-E), once a week during the entire treatment period (14 weeks).

After this procedure, 10 animals of each group were sacrificed and their urinary bladders removed for collection of tissue fragments, which were embedded in paraffin and prepared for histological section, by the usual techniques.

For the histological examinations, sections (2 μm) of the tissue of the urinary bladder were cut using microtome Zeiss, mounted on microscope slides, and stained with hematoxylin-eosin (HE), for pathology assessment, quantification of cellular changes and comparison between control and treatment groups.

Finally, blood from treated and untreated animals was collected at the end of the experiment, processed, and the plasma separated from blood cells was fully digested with the use of concentrated acids (H₂SO₄ and HNO₃) and subjected to techniques for assessing the amount of Mg++, which is the main inorganic component of the molecule (proteic aggregate of ammonium and magnesium phospholinoleate-palmitoleate anhydride) by atomic absorption for verification and quantification of a possible systemic absorption of the compound of the invention from the lining epithelial tissue. This particular assessment, that is, assessment of the amount of Mg++ in the plasma of experimental animals was only performed in the experiments reported in Table 4 (Table 4-Groups A and D).

Criteria Adopted for Assessment of Cellular Changes in Urinary Bladder Cancer and Toxic Effects of Treatments—Table 4, Table 5, Table 6-A, Table 6-B, Table 6-C, Table 6 D

The therapeutic effect in urinary bladder cancer in animals was assessed by histological evaluation of the tissues. The criteria for assessing normality of the tissues examined for all the experiments using the same animal model, that is, the experiments reported in Table 4, Table 5, Table 6-A, Table 6-B, Table 6-C and Table 6-D are: normal tissue, flat hyperplasia, papillary hyperplasia.

Flat hyperplasia and papillary hyperplasia are reactive changes of an inflammatory nature, though benign, and their occurrence in the experiments for assessment of new drugs in animals affected with cancer, indicates a reversal of the cancer process to non-cancerous stages.

The criteria adopted in all the experiments involving urinary bladder cancer in the present report, that is, the experiments contained in tables 4,5 and 6-A, [symbol] 6-B, 6 C, and 6 D, for the presence of malignant cellular changes (cancer) and their degrees of severity are: low grade intraepithelial neoplasia, high grade intraepithelial neoplasia (carcinoma in situ-Tis), papillary carcinoma (Ta) and squamous metaplasia. Metaplasia is defined as the replacement of a normal functional tissue with another tissue with different structure, with loss of function, and, thus, is considered a form of premalignant lesion.

The same criterion was adopted for all experiments involving urinary bladder cancer contained in Tables 4, 5, 6-A [symbol] 6-B, 6-C, and 6-D for assessment of adverse side effects (toxic effect) of the treatments, that is, the presence or absence of hematuria (urinary bleeding) in the animals. In the experiment reported in Table 4, below, this assessment was performed in all animals and compounds used: (proteic aggregate of ammonium and magnesium phospholinoleate-palmitoleate anhydride—topical formulation (Group D-Table 4), injectable formulation (Group E-Table 4) and BCG—topical formulation (Group C-Table 4) and the results are shown in Table 4.

TABLE 4
Histological evaluation

| | | Group B | | Group D | Group E |
|---|---|---|---|---|---|
| | Group A | (MNU) | | (Intravesical | (Injectable |
| | (non treated | Non - | Group C | polymeric | polymeric |
| | control) | treated | (BCG) | anhydride) | anhydride) |
| | n = 10 | n = 10 | n = 10 | n = 10 | N = 10 |
| Flat | — | — | — | 06 (60%) | — |
| hyperplasia | | | | | |
| Papillary | — | 01 = 10% | 4 = 40% | 02 = 20% | 02 (10%) |
| hyperplasia | | | | | |
| Low grade | — | | 02 = 20% | — | 04 (40%) |
| Intraepithelial | | | | | |
| neoplasia | | | | | |
| High-grade | — | 03 = 30% | 02 = 20% | — | 01 (10%) |
| intra-epithelial | | | | | |
| carcinoma | | | | | |
| (carcinoma in | | | | | |
| situ-Tis) | | | | | |
| Papillary | — | 06 = 60% | 01 = 10% | — | 01 (10%) |
| carcinoma (Ta) | | | | | |
| Squamous | — | — | 01 = 10% | — | 02 (20%) |
| metaplasia | | | | | |
| Normal | 100% | — | — | 2 = 20% | — |
| Toxicology: | Negative | — | Positive | Negative | Positive |
| (Hematuria) | (100%) | | (100%) | (0%) | (100%) |
| Mg++/Plasma | 34.6 ± 5.1 | — | — | 32.6 ± 4.2 | — |
| (ug/mL) | | | | | |

Discussion of the Results—Table 4

The therapeutic effect of the topical use of the compound of the present invention (proteic aggregate of ammonium and magnesium phospholinoleate-palmitoleate anhydride), in the practical example provided, that is, treatment of cancer induced in rats by MNU was much higher compared to the best therapy available in the state of the art (BCG) for treating malignant lesions of the epithelium of the urinary bladder.

This therapeutic effect was assessed for all the compounds used, by means of histopathological analysis of the epithelial tissue of the urinary bladder. (Table 4) The experiment showed a positive result for 80% of the animals treated with the compound of the invention, that is, the proteic aggregate of ammonium and magnesium phospholinoleate-palmitoleate anhydride when topically used (Table 4-Group D), with the epithelial tissue of 20% being completely normalized and 60% of the animals showing only flat hyperplasia and 20% with papillary hyperplasia.

However, the use of the immunomodulator (proteic aggregate of ammonium and magnesium phospholinoleate-palmitoleate anhydride-Table 4-Group E), in the injectable form, as subcutaneous injections, at the same concentrations (10 mg/kg) did not show comparable therapeutic effects when used in the topical form, which can be due to the low absorption capacity of the inner tissue of the urinary bladder regarding drugs systemically administered that can only be introduced in the body by parenteral route, as previously described.

The topical use of BGC vaccine showed a positive outcome for 40% of the animals, (Table 4-Group C) that showed papillary hyperplasia, while 20% had high grade neoplasia (carcinoma in situ (Tis)—TNM classification), 20% had low grade intraepithelial neoplasia, 10% had papillary carcinoma (Ta) and 10% of the animals had squamous metaplasia (Table 4-Group C).

Metaplasia, as previously cited, is defined as replacement of a normal functional tissue with another tissue with different structure, with loss of function, and, thus, is considered a form of premalignant lesion.

Regarding the amount of magnesium, the main inorganic component of the proteic aggregate of ammonium and magnesium phospholinoleate-palmitoleate anhydride measured in the plasma of the experimental animals, the quantitative assessment of Mg++ ions showed equal results for the untreated control group (Group A-Table 4) and the treated group (Group D-Table 4), that is, 34.6±5.1 ug/mL. Such data indicates that there was no systemic absorption of the compound of the invention (proteic aggregate of ammonium and magnesium phospholinoleate-palmitoleate anhydride), when topically used (Table 4-Group D).

The non-penetration beyond the epithelial layer (Table 4-group D) is possibly due to the relatively large size of the molecule (proteic aggregate of ammonium and magnesium phospholinoleate palmitoleate anhydride), and/or because of its high molecular weight, which is around 320.000 Dalton (320 kDa).

This above cited finding clearly demonstrates that the biological and antitumor therapeutic effect of the immunomodulator (proteic aggregate of ammonium and magnesium phospholinoleate-palmitoleate anhydride) in the case is only due to a local antitumor effect in the lining epithelium.

Finally, the animals given the carcinogenic compound MNU (Table 4-Group B) and which were not treated with any drug following the application of MNU, almost all of the them (90%) had cancerous lesions, with 60% developing papillary carcinoma (Ta-TNM classification) and 30%, high-grade carcinoma in situ (Tis-TNM classification), indicating that the referred compound (MNU) is highly effective in the experimental induction of cancerous lesions.

Toxicology:

Animals treated with the invention: None of the animals in the experiment that was given the compound used for the purposes of the present invention, that is, the immunomodulator (proteic aggregate of ammonium and magnesium phospholinoleate-palmitoleate anhydride) in continuous topical application (Group D-Table 4) showed signs of urine blood (hematuria).

Animals treated with BCG: In all the animals treated with BCG blood was detected in the urine (hematuria). The degree of hematuria in these animals ranged from moderate (80%) to severe (20%) in response to continuous intravesical application of BCG from the second week. (Group C-Table 4)

Animals treated with injectable immunomodulator (Group E-Table 4): All the animals treated with the immunomodulator (proteic aggregate of ammonium and magnesium phospholinoleate-palmitoleate anhydride) in the injectable form were found to have blood in the urine (hematuria), indicating the low effectiveness of the treatment by this route of administration.

In all the experiments involving the administration of the invention in the treatment of bladder cancer, hematuria was used as parameter and the most evident sign for assessment of the local toxicity of the compounds used, for it is related to chronic irritation or inflammation of the affected tissue (epithelium lining the urinary bladder), by topically applied drugs (e.g. BCG vaccine).

Therefore, the use of the present invention in the practice is also more effective than immunotherapy with BCG regarding toxicity too, since the result of the practical experiment undoubtedly showed that the use of the present invention did not cause any sign of toxicity in the epithelium (Group D-Table 4) compared to BCG (Group C-Table 4) for equal periods and using the same route of administration, i.e. intravesical.

Surprisingly, the compound used for the purposes of the present invention (proteic aggregate of ammonium and magnesium phospholinoleate-palmitoleate anhydride) is not only capable of topical action, which is unprecedented in the state of the art, but is also comparatively more effective in the treatment of bladder cancer than the best therapy available so far in the state of the art, i.e. BCG vaccine.

The results of the use of the invention shown in this example of practical use, when compared to BCG, regarding therapeutic effectiveness and assessment of unwanted side effects, measured for the presence or absence of hematuria, demonstrate that the invention represents a significant advance compared to the therapies in the closest state of the art in the treatment of urinary bladder cancer.

Second Practical Experiment—Use of the Present Invention Compared to Chemothyerapy Drugs in the Intravesical Treatment of Bladder Cancer—(Table-5)

Fifth animals (n=50) were used in the experiment. The therapeutic effect was assessed through histological assessment of tissues, and the toxicological effect of the compounds, through assessment of the presence or absence of urinary bleeding also called hematuria. The results are shown in Table 5.

The following compounds were used in this experiment: proteic aggregate of ammonium and magnesium phospholinoleate-palmitoleate anhydride (PI-0305373-3, US 2006/0093628 A1, EP 1529784 A1), Mitomycin C, Valrubicin, topically administered for comparative assessment in the treatment of MNU-induced cancerous lesions.

All the animals in the experiment were subjected to intravesical applications of 1.5 mg/kg of n-methyl-n-nitrosourea (MNU) dissolved in 0.30 mL of sodium citrate, using a catheter, for 7 weeks. One group (control) of 10 animals was given only saline solution (NaCl 0%), also intravesically and for an equal period.

The purpose of using n-methyl-n-nitrosourea (MNU) was to experimentally induce immunosuppression and subsequently producing urinary bladder carcinoma. After treatment with MNU, the animals were divided into 05 groups of 10 animals each.

After the use of MNU for 7 weeks, 5 batches of 10 animals each were intravesically inoculated with 0.30 ml of 0.9% saline solution (control group), 10 mg/kg of the chemotherapeutic agent Mitomycin C suspended in sterile water for injection, 10 mg/kg of the chemotherapeutic agent Valrubicin, suspended in diluents supplied by the manufacturer and 10 mg/kg of the immunomodulator (proteic aggregate of ammonium and magnesium phospholinoleate-palmitoleate anhydride), suspended in saline solution, using intravesical catheter for the topical application of all the substances during 14 weeks.

After this procedure, all the 10 animals of each group were sacrificed and their urinary bladders were removed for collection of tissue fragments, which were embedded in paraffin and prepared for histological section, by the usual techniques.

For the histological examinations, sections (2 μm) of the tissue of the urinary bladder were cut using microtome Zeiss, mounted on microscope slides, and stained with hematoxylin-eosin (HE), for subsequent pathology assessment, quantification of cellular changes and comparison between control and treatment groups. The criteria for assessment of these lesions, the effects of treatment and the assessment of toxic effects (hematuria) have been previously described.

The comparative therapeutic and toxicity findings regarding the use of the invention, Mitomycin C and Valrubicin are shown in Table-5, below.

TABLE 5
Histological assessment

| | | | Group C | Group D | |
|---|---|---|---|---|---|
| | | | (Mitomycin | Polymeric | Group E |
| | Group A | | D) | anhydride) | (Valrubicin) |
| | (untreated | Group B | n = 10- | n = 10 | N = 10 |
| | control) | (MNU) | topical | topical | topical |
| | n = 10 | n = 10 | application | application | application |
| Flat | — | — | — | 05 (50%) | — |
| hyperplasia | | | | | |
| Papillary | — | 01 = 10% | 4 = 40% | 02 = 20% | — |
| hyperplasia | | | | | |
| Low grade | — | | 03 = 30% | — | 5 (50%) |
| intraepithelial | | | | | |
| neoplasia | | | | | |
| High grade | — | 03 = 30% | 02 = 20% | — | 2 (20%) |
| intraepithelial | | | | | |
| neoplasia | | | | | |
| (carcinoma in | | | | | |
| situ-Tis) | | | | | |
| Papillary | — | 06 = 60% | — | — | 1 (10%) |
| Carcinoma | | | | | |
| (Ta) | | | | | |
| Squamous | — | — | — | — | 1 (10%) |
| metaplasia | | | | | |
| Normal | 100% | — | 01 = 10% | 3 = 30% | 1 (10%) |
| Toxicology: | Negative | — | Positive | Negative | Positive |
| (Hematuria) | (0%) | | (100%) | (0%) | (100%) |

Discussion of the Results—Table 5

Assessment of the data contained in Table-5 clearly shows that the use of the immunomodulator (proteic aggregate of ammonium and magnesium phospholinoleate-palmitoleate anhydride) alone, topically administered, i.e., directly applied (intravesically) to the epithelial tissue lining the urinary bladder, affected by cancer, had a more significant therapeutic effect, either regarding tumor response or lower toxicity (Group D, Table 5), compared to the group treated with the compound Mitomycin C (Group C-Table 5) and also with Valrubicin (Group E-Table 5).

While in the group treated with Mitomycin C only 10% of the animals were within normal parameters, 40% with papillary hyperplasia and 50% still had cancer (Group C-Table-5), in the group treated with intravesical topical application of the compound of the invention 30% of the animals were within normal parameters and 70% had hyperplasia. (Group D-Table 5). In the group treated with Valrubicin (Group E-Table 5) only 10% had tumor regression (normal animals) after treatment.

Since hyperplasia is a non-cancerous condition, based on the experiment reported in Table 5, it can be concluded that the invention had a therapeutic effectiveness of practically 100% (with 70% for hyperplasia and 30% for normality) compared to the control group (Group B-Table 5) in which 90% of the animals had cancer (30% classified as in situ-Tis carcinoma and 60% as papillary carcinoma-Ta).

Regarding Mitomycin C (Group C-Table 5), it showed an efficacy of 40% (Papillary hyperplasia), 50% of the animals had cancer (30% with low-grade carcinoma and 20% with carcinoma in situ-Tis), with only 10% of the animals within normal parameters (Group C-Table 5). Finally the group treated with Valrubicin (Group E-Table 5) showed an effectiveness of only 10% (normal animals).

Regarding toxicology, it is immediately noted that the topical use of the compound of the invention did not result in any local toxicity measured by the presence of hematuria (Group D-Table 5), since none of the animals treated had urinary bleeding (Group D-Table 5), while the animals that used Mitomycin C were 100% positive for the presence of hematuria (Group C-Table 5) as well the animals treated with Valrubicin, i.e., 100% of the animals treated (Group E-Table 5).

Third Practical Experiment—Assessment of the Combined Use of the Invention With Chemotherapy and Immunotherapy Drugs—Table 6-A

Ninety animals (n=90)—Fischer 344 rats were used in the experiment.

The compounds proteic aggregate of ammonium and magnesium phospholinoleate-palmitoleate anhydride (10 mg/kg), BCG (10⁶ CFU (40 mg)) Mitomycin C (10 mg/kg), Valrubicin (10 mg/kg) were used in this experiment, all intravesically administered using a urinary catheter, for comparative assessment in the treatment of MNU-induced cancerous lesions (Table 6-A).

All the animals (Fisher 344 rats), except for the untreated control group (n=10), received intravesical inoculations (1.5 mg/kg) of n-methyl-n-nitrosourea (MNU) dissolved in 0.30 ml of sodium citrate, using a catheter, for 7 weeks. One group (untreated control group) of 10 animals received only saline solution (NaCl 0.9%), also intravesically, and for an equal period. After treatment with MNU, the animals were divided into 07 groups of 10 animals each.

After receiving MNU for 7 weeks, the 9 batches of 10 animals each were subsequently subjected to intravesical inoculation of the compounds of interest during 8 weeks in total.

The groups were divided, as follows: Group A (untreated control) received only saline solution (NaCl at 0.9%) for 8 weeks; group B received only MNU and 0.9% saline; group C received 10 mg/kg of the compound of the invention (proteic aggregate of ammonium and magnesium phospholinoleate-palmitoleate anhydride (10 mg/kg), for 8 weeks, intravesically; group D received BCG vaccine (10⁶ CFU (40 mg)) for 4 weeks, followed by the application of the compound of the present invention (proteic aggregate of ammonium and magnesium phospholinoleate-palmitoleate anhydride—10 mg/kg) for another 4 weeks.

Group E received Mitomycin C (10 mg/kg) for 4 weeks, followed by the application of the compound of the present invention (proteic aggregate of ammonium and magnesium phospholinoleate-palmitoleate anhydride 10 mg/kg) for another 4 weeks; group F received BCG vaccine (10⁶ CFU (40 mg)) for 8 weeks and group G received Mitomycin C (10 mg/kg) for 8 weeks. Group H received Valrubicin (10 mg/kg) for 8 weeks and Group I received Valrubicin (10 mg/kg) for 4 weeks, followed by application of the compound of the present invention (proteic aggregate of ammonium and magnesium phospholinoleate-palmitoleate anhydride (10 mg/kg) for another 4 weeks.

All the compounds were topically administered, by intravesical instillation, in the animals, using the appropriate diluents already described in the present report. For better comprehension of the experiment, see the table below, called Experimental Groups.

| Table Experimental Groups | Compounds and periods of use |
|---|---|
| Group A (n = 10) | Saline 0.9%- Untreated control group |
| Group B (n = 10) | MNU + saline 0.9% - 8 weeks |
| Group C (n = 10) | proteic aggregate of ammonium and |
| | magnesium phospholinoleate-palmitoleate |
| | anhydride for 8 weeks (10 mg/kg) |
| Group D (n = 10) | BCG vaccine (10⁶ CFU (40 mg)) for 4 |
| | weeks |
| | proteic aggregate of ammonium and |
| | magnesium phospholinoleate-palmitoleate |
| | anhydride for 4 weeks (10 mg/kg) |
| Group E (n = 10) | Mitomycin C (5 mg/kg) for 4 weeks |
| | proteic aggregate of ammonium and |
| | magnesium phospholinoleate-palmitoleate |
| | anhydride for 4 weeks (10 mg/kg) |
| Group F (n = 10) | BCG vaccine (10⁶ CFU (40 mg)) for 8 |
| | weeks |
| Group o G (n = 10) | Mitomycin C (10 mg/kg) for 8 weeks |
| Group H (n = 10) | Valrubicin (10 mg/kg) for 8 weeks |
| Group I (n = 10) | Valrubicin (10 mg/kg) -4 weeks + |
| | proteic aggregate of ammonium and |
| | magnesium phospholinoleate-palmitoleate |
| | anhydride (10 mg/kg) for 4 weeks |

After these procedures, and at the end of the experiment, all the 10 animals of each group were sacrificed, and their urinary bladders were removed for collection of tissue fragments, which were embedded in paraffin and prepared for histological section, by the usual techniques, previously described in the present report.

The comparative results of the use of the compound of the invention used in combination or association with other compounds and compared to the isolated results for each compound are shown in Table 6-A.

The criteria for assessment of lesions, effects of treatment and assessment of toxic effects (hematuria) have been previously described.

TABLE 6-A

| | | Group | | | Group E | | | | |
|---|---|---|---|---|---|---|---|---|---|
| | | B | | Group D | n = 10 | | | | |
| | Group A | n = 10 | Group C | n = 10 | Mitomycin | Group | Group G | | Group I |
| | n = 10 | MNU + | n = 10 | BCG + | C + | F | n = 10 | Group H | Valrubicin + |
| Histological | Untreated | saline | Polymeric | Polymeric | Polymeric | n = 10 | Mitomycin | N = 10 | Polymeric |
| assessment | control | 0.9% | anhydride | anhydride | anhydride | BCG | C | Valrubicin | anhydride |
| Flat | — | — | 4(40%) | 4(40%) | 3(30%) | 2(20%) | 1(10%) | 1(10%) | 40(%) |
| hyperplasia | | | | | | | | | |
| Papillary | — | — | 3(30%) | 3(30%) | 4(20%) | 2(20%) | | 1(10%) | 4(40%) |
| hyperplasia | | | | | | | | | |
| Low-grade | — | 4(40%) | | 1(10%) | 1(20%) | 3(30%) | 6(60%) | 4(40%) | 1(10%) |
| intraepithelial | | | | | | | | | |
| carcinoma | | | | | | | | | |
| High-grade | — | 5(50%) | | | | 2(20%) | 3(30%) | 2(20%) | |
| intraepithelial | | | | | | | | | |
| carcinoma | | | | | | | | | |
| (carcinoma | | | | | | | | | |
| in situ-Tis) | | | | | | | | | |
| Papillary | — | 1(10%) | | | | | | 1(10%) | |
| carcinoma | | | | | | | | | |
| (Ta) | | | | | | | | | |
| Squamous | — | — | | | | 1(10%) | | 1(10%) | |
| metaplasia | | | | | | | | | |
| Normal | 100% | 0% | 3(30%) | 2(20%) | 2(20%) | 0% | 0% | — | 1(10%) |
| Toxicology: | Negative | — | Negative | Positive | Positive | Positive | Positive | Positive | Negative |
| Presence of | (0%) | | (0%) | n = 2 | n = 2 | n = 10 | n = 5 | N = 10 | |
| hematuria | | | | (20%) | (20%) | (100%) | (50%) | 100% | |

Table 6-A—Discussion of the Results

Analysis of data shown in Table-6-A reveals that the topical use of the invention in intravesical instillation associated with other drugs (Mitomycin C, Valrubicin and BCG vaccine) not only showed synergistic effects (Group D, Group E, Group I-Table 6-A), or else, maximized the therapeutic effects, but also significantly reduced unwanted side effects (hematuria) compared to the isolated use (Groups F, G, H-Table 6-A) of the same compounds (BCG, Mitomycin C, Valrubicin).

Based on the data shown in Table 6-A, any person skilled in the art easily understands that the invention also can replace the above mentioned drugs in the case of failure or occurrence of side effects that prevent their use.

It can be also seen that the use of the invention has notably contributed to reduce local toxicity (hematuria) when combined with Mitomycin C and BCG vaccine (Group D and Group E-Table 6-A) and also with Valrubicin (Group I-Table 6-A).

Also, information on the practical use of the invention, notably synergy with other drugs, i.e. maximization of therapeutic effects associated with the reduced toxicity of the combination (Group D, Group E, Group I-Table 6-A), clearly demonstrates the benefits of its use in clinical practice, i.e., in protocols combined with chemotherapy and immunotherapy drugs in the topical treatment of cancer affecting the inside and outside epithelium (Table-6-A-Group D, Group E, Group I) with the purpose of maximizing the effectiveness of the combined therapy and simultaneously minimizing or neutralizing inflammatory disorders, such as hematuria associated to the cancerous process and the toxicity of the drugs.

These data concerning the practical use of the invention, notably synergy, demonstrated by the maximization of the therapeutic effects when associated to other drugs, including reduction in toxicity, allow to forecast its use in clinical practice for the palliative treatment of cancer of the epithelial tissue lining inside or outside surfaces of the body, particularly the urinary bladder and uterus, when surgical treatments are no feasible due to medical problems related to high probability of unacceptable morbidity and/or high risk of mortality (Table 6-A).

Formulations for the Invention in the Treatment of Epithelial Cancer

As already cited, in the current state of the art, the best therapy for tumors of the epithelial tissue is based on surgical removal of the lesions, when feasible, followed by immediate topical application of chemotherapy or immunotherapy drugs.

For the successful deposition or use of these compounds, it is necessary to develop pharmaceutical formulations capable of ensuring their solubilization and facilitating their adherence or concentration on cancerous tissues.

For the purposes of the present invention, the immunomodulator (proteic aggregate of ammonium and magnesium phospholinoleate-palmitoleate anhydride) was used in the form of aqueous suspension, a sterile solution of sodium chloride 0.9% in water (NaCl 0, 9%) in experiments of the practical use of topical treatment of cancer in the lining epithelium (urinary bladder), as described in Tables 4, 5 and 6-A, 6-B, 6-C, 6-D.

As described in the present report, in the treatment of male patients, the immunomodulator (proteic aggregate of ammonium and magnesium phospholinoleate-palmitoleate anhydride) was also used in the form of a topical cream at pH 7, with 15% concentration of the active ingredient, using sterile vaseline (petroleum jelly) and sterile deionized distilled water as carriers (Table 7). For the treatment of premalignant lesions in female patients, the immunomodulator (proteic aggregate of ammonium and magnesium phospholinoleate-palmitoleate anhydride) was formulated using a topical cream with 20% concentration of the active ingredient (Table 8-A and Table 8-B).

For all these formulations, either using saline solution (NaCl 0.9%) used in the experiments shown in Table 4, Table 5 and Table 6-A, 6B, 6-C, 6-D, Table 9 and also creams for topical application used in the experiments shown in Table 7, Table 8A and Table 8B, the invention was able to effectively fight malignant and premalignant lesions in the epithelial tissue lining surfaces inside the body (urinary bladder, uterus) and outside (skin) at the referred doses.

However, the immunomodulator (proteic aggregate of ammonium and magnesium phospholinoleate-palmitoleate anhydride) is a poorly soluble drug. Thus, in order to increase its solubility and improve its penetration in the epithelium, some pharmaceutically acceptable carriers were selected and tested in combination with the immunomodulator, including polymers, polyethylene glycols, polyethylene oxide (PEO), non-ionic surfactants and edible oils.

In the state of art, some polymers such as polyethylene-polypropylene-glycol, also named Polaxamers are reported as able to induce functional alterations in cells. In addition, Poloxamers have been reported as able to preferentially target cancer cells, due to differences in the membrane of these cells when compared to noncancerous cells. For reference: Batrakova et al. Pluronic block copolymers: Evolution of drug delivery concept from inert nanocarriers to biological response. Journal of Controlled Release 130 (2008) 98-106doi:10.1016/j.jconrel.2008.04.013

As a result, the Poloxamers may cause important biological effects such as sensitization of tumors to various anticancer agents. For reference: Batrakova et al. Pluronic block copolymers: Evolution of drug delivery concept from inert nanocarriers to biological response. Journal of Controlled Release 130 (2008) 98-106doi:10.1016/j.jconrel.2008.04.013.

For all the reasons above cited, a Polaxamer (polyethylene-polypropylene-glycol (CAS registry number 9003-11-6) Molecular formula: HO. (C2H4O) m. (C3H6) n.H) was specifically selected to be tested as a possible pharmaceutical carrier, for the immunomodulator (proteic aggregate of ammonium and magnesium phospholinoleate palmitoleate anhydride) in an animal model for the study of urinary bladder cancer (Table 6-B).

The results obtained in animal model with the use of the immunomodulator (proteic aggregate of ammonium and magnesium phospholinoleate-palmitoleate anhydride) with its formulation, using polyethylene-polypropylene-glycol, used as pharmaceutical carrier, when compared to the application of the immunomodulator (proteic aggregate of ammonium and magnesium phospholinoleate-palmitoleate anhydride) using saline solution (sodium hydrochloride —NaCl 0.9%) as pharmaceutical carrier were absolutely outstanding and unprecedented in the state of the art.

As shown in Table 6-B with a formulation using polyethylene-polypropylene-glycol as pharmaceutical carrier, for the same therapeutic effect in urinary bladder cancer, a 10 times lower amount of the active ingredient (proteic aggregate of ammonium and magnesium phospholinoleate-palmitoleate anhydride) was required (Table 6B-Group E).

Example of Practice Use for Comparative Purposes, Using Compound of the Invention in Saline Solution, and With Addition in Polymer (Poloxamer)—Table 6-B

Fifty (50) Fischer 344 rats, aged 7 weeks, weighing in average 150 grams were used in this experiment.

All the animals (Fischer 344 rats), except for the untreated control group (Table 6B-Group A), were subjected to applications of 1.5 mg/kg of n-methyl-n-nitrosourea (MNU) dissolved in 0.30 ml of sodium citrate, intravesically, using a catheter (for insertion in the bladder) for 7 weeks for bladder cancer induction. After administration of MNU the animals were divided into 5 groups of 10 animals each.

One group (untreated control) of 10 animals was given only saline solution (NaCl 0.9%) also intravesically and for an equal period (Table 6B-Group A). One group equally induced with MNU (Table 6-B Group B) did not receive any treatment for comparison purposes and assessment of lesions.

After the period of cancer induction the animals were treated with the compounds proteic aggregate of ammonium and magnesium phospholinoleate-palmitoleate anhydride at the dose of 10 mg/kg (Table 6B-Group C) added to saline solution (NaCl 0.9%), the compound proteic aggregate of ammonium and magnesium phospholinoleate-palmitoleate anhydride at the final dose of 1 mg/ml mg/kg (GROUP D) also added to saline solution (NaCl 0.9%).

Finally, after the period of cancer induction, the animals were also treated with the immunomodulator (proteic aggregate of ammonium and magnesium phospholinoleate-palmitoleate anhydride) at the dose of 0.2 mg dissolved in 0.2 mL of aqueous solution of Poloxamer (polyethylene-polypropylene-glycol) or else, at the final concentration of 1 mg/mL, (GROUP E).

All the compounds were intravesically inoculated, with a catheter (for insertion into the bladder) for this purpose, for 6 consecutive weeks, for comparative assessment in the treatment of MNU-induced cancerous lesions.

The results are shown in Table 6-B below.

The comparative results of the use of the compound of the invention in saline solution (NaCl 0.9%) at the concentration of 10 mg/kg (Table 6B-Group C), saline solution at the concentration of 1 mg/kg (Table 6B-Group D) and finally of the immunomodulator (proteic aggregate of ammonium and magnesium phospholinoleate-palmitoleate anhydride) at the dose of 0.2 mg dissolved in 0.2 mL of aqueous solution of Poloxamer (polyethylene-polypropylene-glycol) or else, at final concentration of 1 mg/mL (Table 6-B-Group E) are shown in Table 6-B below.

The criteria used for assessment of the lesions, effects of treatment and assessment of toxicity (hematuria) for the experiments of Table 6-B have been previously described (see specification)

TABLE 6-B
Histological assessment

| | | | Group C | Group D | |
|---|---|---|---|---|---|
| | Group A | Group B | Immunomodulator + | Immunomodulator + | Group E |
| | n = 10 | n = 10 | saline solution | saline solution | Immunomodulator + |
| | Untreated | MNU + saline | at 0.9% | at 0.9% | Poloxamer |
| | control | solution | 10 mg/kg | 1 mg/ml | 1 mg/ml |
| | group | at 0.9% | intravesical | intravesical | intravesical |
| Flat | — | — | 2 (20%) | — | 4 (40%) |
| hyperplasia | | | | | |
| Papillary | — | — | 6 (60%) | 1 (10%) | 2 (20%) |
| hyperplasia | | | | | |
| Low-grade | — | 1 (10%) | — | 2 (20%) | 1 (10%) |
| intraepithelial | | | | | |
| carcinoma | | | | | |
| High-grade | — | 3 (30%) | — | 1 (10%) | — |
| intraepithelial | | | | | |
| carcinoma | | | | | |
| (carcinoma | | | | | |
| in situ-Tis) | | | | | |
| Papillary | — | 4 (40%) | — | 1 (10%) | 1 (10%) |
| carcinoma | | | | | |
| (Ta) | | | | | |
| Squamous | — | 2 (10%) | 1 (10%) | 5 (50%) | — |
| metaplasia | | | | | |
| Normal | 100% | — | 1 (10%) | — | 2 (20%) |
| Toxicology: | 0% | 10 (100%) | 0% | 4 (40%) | 0% |
| Presence of | | | | | |
| hematuria | | | | | |

Discussion of the Results

The therapeutical results for intravesical use of the immunomodulator (proteic aggregate of ammonium and magnesium phospholinoleate-palmitoleate anhydride) at the dosage of 10 mg/kg (Table 6B-Group C), by intravesical instillation, in topical form, suspended in saline solution (NaCl 0.9%) are similar to those shown in Table 4.

Or else, with the topical use (10 mg/kg—Group C-Table 6-B) 90% of tumor reversion was obtained (Group C-Table 6-B) compared to the animals induced with MNU and treated only with saline solution (NaCl 0.9%), where malignant and premalignant lesions were found in all of them (100%) (Group B-Table 6-B).

The absolute novelty compared to the previous state is that the use of the immunomodulator (proteic aggregate of ammonium and magnesium phospholinoleate-palmitoleate anhydride) at the dose of 0.2 mg dissolved in 0.2 mL of aqueous solution of de Poloxamer (polyethylene-polypropylene-glycol) or else, at final concentration of 1 mg/mL (Table 6-B-Group E) was equally effective in the treatment of cancerous lesions in the epithelium of the urinary bladder (Group-E-Table 6-B) with therapeutic results comparable to those of Group C-Table 6-B that used a dose (10 mg/kg) of the immunomodulator (proteic aggregate of ammonium and magnesium phospholinoleate-palmitoleate anhydride) 10 times greater to obtain a similar therapeutic effect.

The topical use of the immunomodulator (proteic aggregate of ammonium and magnesium phospholinoleate-palmitoleate anhydride(Group D-Table 6-B) at the dose of 1 mg/kg, dissolved in saline solution (NaCl 0.9%) did not show therapeutic effects compared to those observed in Group C-Table 6-B and in Group E-Table 6-B.

The experiment of the practical use of the invention demonstrates that the pharmaceutical formulation of the immunomodulator (proteic aggregate of ammonium and magnesium phospholinoleate-palmitoleate anhydride) with Poloxamer (polyethylene-polypropylene-glycol) was capable of producing unprecedented practical effects, measured by a remarkable decrease in the required amounts of the active ingredient (proteic aggregate of ammonium and magnesium phospholinoleate-palmitoleate anhydride) for the treatment of tumors in the epithelial tissue, in the specific case, those located in the urinary bladder.

Any person skilled in the art understands that a formulation that allows reducing the quantities of drugs required to obtain a therapeutic effect has undeniable economic advantages, and also in what concerns safety, due to the smaller number of substances to be deposed in the body.

Therefore, the use of the Poloxamer (polyethylene-polypropylene-glycol) for the elaboration of formulations with the immunomodulator (proteic aggregate of ammonium and magnesium phospholinoleate-palmitoleate anhydride) represents a clear improvement in the state of the art, for the use of this compound in the situations in which it is required, as shown in the present invention.

Cancer in the Epithelial Tissue Lining Surfaces Outside the Body—Skin Cancer—State of the Art

Introduction

Like any other type of cancer, skin cancer is broadly defined as the uncontrolled growth of cells that have the potential to spread to or penetrate into other parts of the body, which usually occurs if such lesions are not detected and treated in a timely manner.

Skin cancer can be caused by many factors, one of the major factors being over exposure to UV radiation from the sun. It is considered the most common type of cancer, corresponding to 25% of all neoplastic lesions.

Other causes include prolonged exposure to chemicals, particularly the action of some types of viruses such as the human papilloma virus or HPV, genetic factors and finally low immunity.

Regarding human papilloma viruses (HPV), these agents are listed as causes of potentially malignant causes, also called cell dysplasias in the epithelial tissue lining surfaces inside or outside the body.

The papilloma viruses or human papilloma viruses (HPV) cause several clinical manifestations such as skin warts, genital warts and finally premalignant cell changes (dysplasias) in the epithelium lining the cervix, which often can develop into cancer if left untreated.

In the state of the art, the treatments of lesions caused by HPV viruses in the epithelium lining surfaces outside the body (skin) involve elimination or surgical removal of the lesions, such as abrasion treatment, laser surgery and cryocauterization, for example.

However, the excision or elimination of the affected tissue alone is not is not able to prevent recurrence of the tumor or lesions caused by the virus in most occasions, and therefore the use of combined therapies is needed.

With the advance of the state of the art, some new drugs, notably immunomodulators such as Interferons, and the compound called Imiquimod, are now used as a topical adjuvant therapy against cutaneous forms of HPV associated to or preceded by previous surgical removal of the lesions.

In general, it can be said that the selection of the type of treatment of premalignant or malignant lesions will depend on the type of cancer, location of the cancer, patient's age and on whether the lesion is a primary cancer or a recurrence.

In the event of a cancer already manifested or diagnosed, in the state of the art, the typical treatment is surgical removal of lesions using different techniques such as abrasion treatment, laser surgery, cryocauterization, etc, followed by radiotherapy or chemotherapy with topical action. For example, surgery followed by topical chemotherapy may be indicated for the treatment of superficial basal cell carcinoma.

For low-risk tumors, in some cases, external radiation or topical chemotherapy with compounds as imiquimod, resiquimod and the compound called 5-fluorouracil and other procedures such as cryotherapy may be suitable.

Other types of treatment such as photodynamic therapy, with compounds that activate with polarized light or laser can also be used in combination with surgical excision of the tumors.

Other more aggressive epithelial cancers such as melanoma, which is considered the most lethal type of skin cancer, are less sensitive to radiation or chemotherapy, but can be fought with a reasonable degree of success due to the combination of surgical excision of tumors followed by the application of topical or systemic immunotherapy, particularly if detection of malignant or premalignant lesions is made early.

In short, it is well established in the state of the art that the best treatment for malignant or premalignant lesions affecting the epithelial tissue lining outside the body (skin) should include a combination of therapies, particularly those with topical action, to maximize therapeutic effectiveness and minimize the occurrence of relapses.

The same understanding applies for malignant or premalignant lesions affecting the epithelial tissue lining surfaces inside the body, e.g. bladder cancer, esophagus cancer and premalignant or malignant uterine lesions caused by several types of HPV.

The present invention, due to the novel and unprecedented functionalities of the immunomodulator (proteic aggregate of ammonium and magnesium phospholinoleate-palmitoleate anhydride) presented in this report is especially suitable to meet these requirements and also represents a novel and remarkable advance in the state of the art for the topical treatment of malignant lesions in the epithelial tissue lining outside and inside surfaces of the body.

For illustration purposes, two examples of the practical use of the invention in the topical use of lesions associated to HPV virus affecting the epithelial tissue lining surfaces outside (skin) and inside (uterus) the body are provided.

These practical examples are merely illustrative and do not intend to reduce or limit the field of application or the scope of the present invention.

Besides indicating the effectiveness of the invention in the treatment of these specific diseases, the results obtained in these experiments of practical use also demonstrate the usefulness of the invention for other situations that may require topical treatment against malignant or premalignant lesions in the epithelial tissue lining outside or inside surfaces of the body.

Example of Practical Use—Use of the Invention for the Topical Treatment of Genital Warts Associated to Virus (HPV)—Table-7

For this experiment of the practical use of the invention, 10 volunteers were selected, all of them male individuals, with genital warts epithelium, clinically diagnosed as condylomata acuminata also named genital warts.

Genital warts (condylomata acuminata) are clinical symptoms of a highly contagious sexually transmitted disease caused by some types of human papillomavirus (HPV). It is very difficult to treat genital warts. Existing treatments are focused on the removal of visible warts, but these may also regress. The reason is that although treatments can remove the warts, but frequently they do not remove the HPV, so warts can recur after treatment. Furthermore, such treatments are often toxic and very painful. For reference: Geo von Krogh, Eric Longstaff Podophyllin office therapy against condyloma should be abandoned. Sex Transm Infect 2001; 77:409-412 doi:10.1136/sti.77.6.409

All patients were admitted for treatment with the compound of the invention showing recurrence of cancer after previous topical treatment of genital warts with podophyllin.

Laboratory tests of tissue samples from the lesions of all patients, prior to the treatment, were analyzed by in situ hybridization technique and found to be positive for HPV type 11), confirming the clinical diagnosis of condylomata acuminata or genital warts of the penile region associated to HPV.

Of the 10 voluntary patients, 6 (patients 1, 2, 3, 4, 5 and 6) were subjected to excision of the genital warts with topical application of Podophyllin in the affected regions for 4 days, with an interval of 2 days, followed by daily topical application for each patient of the compound of the present invention (proteic aggregate of ammonium and magnesium phospholinoleate-palmitoleate anhydride) suspended in appropriate vehicle (cream for topical application-pH 7) composed of sterile vaseline and deionized sterile distilled water with 15% concentration of the active ingredient (proteic aggregate of ammonium and magnesium phospholinoleate-palmitoleate anhydride) for 4 more days.

Of the 10 patients, 4 (patients 7, 8, 9,10) were subjected only to the previous treatment, i.e., excision of genital warts with topical application of podophyllin for 8 days in total, without the subsequent administration of the compound of the present invention.

The participants were monitored for 30 more days for assessment of possible recurrence of lesions and again subjected to laboratory analysis with in situ hybridization technique to evaluate the possible presence of HPV.

Results

Remarkable results were obtained with the topical use of the compound of the present invention. Of the patients who used the combined treatment (patients 1, 2, 3, 4, 5, 6), only one patient (Table 7-patient 2) showed recurrence of lesions, i.e. new reappearance of lesions (warts or condyloma) in the penile region.

Post-treatment laboratory test (in situ hybridization) of this patient (patient 2) was also positive for the presence of HPV.

All the other patients (patients 1, 3, 4, 5, 6), did not show recurrence of lesions (warts or condyloma) in a post-treatment clinical and laboratory follow-up.

The patients without recurrence who used the compound of the present invention (patients 1, 3, 4, 5, 6) were also negative for the presence of HPV when subjected to specific laboratory test (in situ hybridization).

All the patients who were given only Podophyllin (patients 7, 8, 9,10) were found to be positive for a new recurrence of genital warts associated with the residual presence of HPV virus detected in specific laboratory test (in situ hybridization).

TABLE 7

| | | | | Application of | | |
|---|---|---|---|---|---|---|
| | | | | Podophyllin | | |
| | | Pre- | | (4 days) + | | Post- |
| | Clinical | treatment | Application | immunomodulator | Clinical | treatment |
| | presentation: | in situ | of | in topical | presentation:: | in situ |
| | Relapsed | hybridization | Podophyllin | form (4 days) + | Recurrent | hybridization |
| Patients | condyloma | for HPV | (8 days) | (4 days) | condyloma | for HPV |
| Patient 1 | positive | positive | no | yes | negative | negative |
| Patient 2 | positive | positive | no | yes | positive * | positive* |
| Patient 3 | positive | positive | no | yes | negative | negative |
| Patient 4 | positive | positive | no | yes | negative | negative |
| Patient 5 | positive | positive | no | yes | negative | negative |
| Patient 6 | positive | positive | no | yes | negative | negative |
| Patient 7 | positive | positive | yes | no | positive | positive |
| Patient 8 | positive | positive | yes | no | positive | positive |
| Patient 9 | positive | positive | yes | no | positive | positive |
| Patient 10 | positive | positive | yes | no | positive | positive |

Discussion of the Results—Table 7

It can be concluded that the use of the compound of the present invention, topically applied to the epithelium, showed a clinical and laboratory efficacy of approximately 90% (Table-7), which undoubtedly represents a remarkable gain in effectiveness, compared to one of the main types of treatment in the state of the art, for the topical treatment of this type of lesion associated to the presence and action of HPV virus.

Although in this case the procedure used was removal of the genital warts, after excision of the lesions by chemical means, i.e. with the use of podophyllin, the invention can also be used in combination and association with other therapies or techniques, as, for example, removal by cryocauterization or else electrocauterization and other topical surgical procedures.

In conclusion, this experiment of practical use demonstrates that the invention can be advantageously used as adjuvant therapy for the treatment for premalignant lesions, notably those caused by aggressive agents, e.g. genital warts related to HPV in the epithelial tissue lining outside surfaces, associated to other existing procedures in the state of the art, such as surgical procedures.

Premalignant Lesions of the Uterine Cervix and Evolution to Cancer—Associated Factors—Treatments—State of the Art

The so-called cervical dysplasia is considered a premalignant or precancerous change in epithelial cells of the cervix. For illustrative purposes, we cite Kevin A. Ault-Epidemiology and natural history of human papillomavirus infections in the female genital tract. Infect Dis Obstet Gynecol. 2006; 2006 Suppl: 40470. Infect Dis Obstet Gynecol. 2006; 2006:40470.

In some cases, cervical dysplasia remains stable or is eliminated or reversed by the immune system. However, if not timely treated, it may become a chronic condition and progress to cervical cancer.

Cervical dysplasia is routinely diagnosed in clinical practice with the Papanicolaou or Pap-smear test, which is used for early detection of the lesions.

The so-called Pap-smear test is a routine gynecological test with cells collected from the cervical region (cells from the uterine cervix). It shows signs related to infections in the region, as well as malignant lesions.

Besides its classical use for assessment of cells of the cervix, the test is also used to evaluate the effect of treatments.

The result of Pap smears is grouped into 5 (five) classes: Class I means absence of abnormal cells, Class II generally indicates inflammation or infection and Class III indicates the presence of abnormal cells (dysplasia).

Class III can be subdivided into three subclasses or types of dysplasia: mild, moderate or severe.

Cauterization is usually indicated in mild and moderate dysplasia. In severe dysplasia, a surgery to remove a cone-shaped piece of tissue from the cervix (conization) or other surgical measures may be needed.

Class IV dysplasia is a highly suspicious finding for malignancy and Class V indicates manifestation of cervical cancer.

Determinant Causes for Cervical Dysplasia—Chronic Infection by HPV

It is well established in the state of the art that the determining factor for most chronic cervical dysplasias, which if not effectively treated may lead to cervical cancer, is the infection caused by the sexually transmitted Human Papilloma virus—HPV.

There are approximately 100 types of identified HPV, of which about a dozen are involved in the process that generates cervical dysplasia and its progression to cervical cancer. HPV-16 and HPV-18 types are responsible for 60% of the cases of cervical cancer worldwide. Munoz N, Castellsague X, de Gonzalez A B, Gissmann L. Chapter 1: HPV in the etiology of human cancer. Vaccine. 2006; 24 Suppl 3:S1-S10. Rositch A F et al. The incidence of human papillomavirus infection following treatment for cervical neoplasia: a systematic review. Gynecol Oncol. 2014 March; 132(3):767-79.

HPV 6, HPV-11, HPV-16, HPV-18, HPV-33 and HPV-45 are high-risk/carcinogenic HPV types. For an example of the state of art, we cite: Rositch A F et al. The incidence of human papillomavirus infection following treatment for cervical neoplasia: a systematic review. Gynecol Oncol. 2014 March; 132(3):767-79.

These types cited (HPV 6, HPV-11, HPV-16, HPV-18, HPV-33, HPV-45) and recognized as being of high risk are associated to the development of cervical dysplasias, i.e. precancerous cell lesions, as they may progress to cancer. The presence and characterization of these viral types are assessed in the state of the art, following diagnosis of cervical dysplasia through several laboratory techniques such as in situ hybridization and PCR (Polymerase chain reaction).

There is no evidence yet in the state of the art of whether cell changes called dysplasias (cervical dysplasia) may be directly associated to viral activity (direct cytopathogenic effect) or indirectly associated due to cell changes induced by host-pathogen interaction (indirect cytopathogenic effect) or a combination of these causes.

However, regardless of the causal factors of cervical dysplasia, any expert with knowledge of the state of the art can easily understand that the concomitant treatment of the primary carcinogenic cause, that is, chronic viral infection, prior to, associated with or subsequent to treatment or removal of dysplasia, which is a lesion usually associated to the presence of HPV, is important for a successful therapy because it reduces the chance of recurrence of the infection that can lead to dysplasia again.

A hypothetical ideal treatment of a precancerous condition or cellular change, that is, cervical dysplasia, which is associated to chronic infection by HPV, should attack or remove abnormal cells or cause lesions to regress to less aggressive classes according to the Papanicolaou (Pap) classification, and at the same time contribute to the control of the initial causative agent, that is, HPV. As will be shown in the present report, this can be obtained with the present invention.

Cervical Dysplasia—Preventive Therapies—Options of Treatment of Causal Agents (HPV)

Since it has been proven that chronic infection by HPV may induce dysplasias and progression of this condition to cancer, various treatments were developed and are available in the state of the art to attempt to control and/or eliminate the viruses and associated lesions and/or at least to provide a palliative treatment of the dysplasias and cancer. For examples of the state of the art: Scheinfeld N, Lehman D S. An evidence-based review of medical and surgical treatments of genital warts. Dermatol Online J. 2006 Mar. 30; 12(3):5. Retrieved from: http://escholarship.org/uc/item/7v57p744

Infection by HPV can occur in three main presentation forms: clinical, subclinical and latent: The clinical form, with the presence of macroscopic lesions (genital warts) in the anogenital region; the subclinical form, characterized by the presence of diffuse epithelial hyperplasia and dysplasia, seen through the colposcope and after application of contrast medium (acetic acid) and the latent form, that is, without histological changes, though with the presence of the viral DNA detected by techniques such as hybridization, hybrid capture or PCR (polymerase chain reaction).

The traditional treatments aimed mostly to the elimination of lesions associated to HPV or its clinical manifestations, such as genital warts, involve topical therapies, with the use of corrosive agents such as podophyllin and its derivatives, and also trichloroacetic acid. Such treatments are often toxic and painful. For reference: Geo von Krogh, Eric Longstaff. Podophyllin office therapy against condyloma should be abandoned. Sex Transm Infect 2001; 77:409-412 doi:10.1136/sti.77.6.409

Several surgical techniques are also used with varying degrees of success in the state of the art: local excision, cryotherapy, CO2 laser vaporization and electrocauterization. However, the surgical treatments for the clinical and subclinical forms of HPV are not very efficient and have a high degree of recurrence. They involve long periods of time and are often painful and/or disfiguring.

Problems Involving Surgical Treatments for the Elimination of Dysplasias

Surgical procedures to remove HPV infected tissues can be painful and impractical for the treatment of extensive lesions. In the state of the art there is great controversy as to whether the frequent post-treatment recurrences are due to reactivation of subclinical infection or derived from normal epithelium left untreated. For examples of the state of the art, we cite: Krebs, H.-B., 1989. Management strategies. Clinical Obstetrics and Gynecology. 32(1): 200-213 and Leszczyszyn J et al. Anal warts (condylomata acuminata)—current issues and treatment modalities. Adv Clin Exp Med. 2014 March-April; 23(2):307-11.The most frequent complications include pain, local secretion, ulceration, infection and delayed healing. Permanent scarring may also occur.

[symbol]

[symbol]

[symbol]

[symbol]

Recent Advances in the State of the Art—Immunotherapy

In the state of the art, the use of immunotherapy for the treatment of HPV infection and the damage caused by this virus to the cells (dysplasias) started with BCG vaccine, also used in tuberculosis prophylaxis treatment, and with exogenous interferons (e.g. Interferon-alpha, interferon-beta, interferon-gamma), topically or intralesionally applied, to increase immunity. Locally used interferon appears to be much more effective than systemically used interferon. For examples of the state of the art, we cite: Yang J et al. Interferon for the treatment of genital warts: a systematic review. BMC Infect Dis. 2009 Sep. 21; 9:156. doi: 10.1186/1471-2334-9-156.

With the development of new compounds, immunotherapy becomes an attractive therapeutic option in the state of the art for the treatment of HPV infections. Among the new compounds available in the state of the art, we cite the immunomodulator called Imiquimod: (IUPAC: -(2-methylpropyl)-3,5,8-triazatricyclo [7.4.0.02,6]trideca-1(9), 2(6),4,7,10,12-hexaen-7-amine).

Advances in the State of the Art—Prophylactic and Therapeutic Vaccines Against HPV

The most recent advances in the state of the art concern the development of vaccines against HPV, which act in a preventive way against the main carcinogenic types of HPV (HPV-6, HPV-11, HPV-16 and HPV-18). They are only efficient for women who have not yet reached sexual maturity or with no prior infection with HPV. There is not yet a therapeutic vaccine against HPV that ensures its elimination in patients already infected or with a previous history of infection. For reference: Kuang S K et al. Current status of human papillomavirus vaccines. Clin Exp Vaccine Res. July 2014; 3(2): 168-175.

Treatment of Precancerous Lesions and Cervical Dysplasias—State of the Art

In the state of the art, it is widely accepted that the first stage of development of cervical cancer is the condition known as dysplasia, which occurs when squamous cells in the cervical region become abnormal in size and shape and begin to multiply.

Cervical dysplasia (III degree Papanicolaou) is classified as mild, moderate or severe, depending on the degree of cellular abnormality on microscopic examination.

In mild dysplasia, abnormal cells appear only on the surface layer of the epithelium. This condition may progress if not timely treated, evolving to severe dysplasia.

If not treated, severe dysplasia will progress in most cases to the maximum stage or degree, which is early cervical cancer, also known as in situ carcinoma (Tis-TNM classification).

Chemical abrasion with the use of corrosive chemicals, such as Podophyllin or Podophyllotoxin, and surgical procedures performed by mechanical, electrical (cauterization) and freezing (cryosurgery) are used to eliminate and/or remove cervical epithelial lesions usually associated to the action of the human papilloma virus (HPV).

Regardless of the techniques used to remove tissue damaged or changed, it is evident for any expert with knowledge of the state of the art that the success of preventive, curative or palliative treatments of precancerous lesions, that is, cervical dysplasias of any nature, with progression to cancer avoided, is also influenced by the efficient treatment of the main causative agent, that is, HPV, particularly the carcinogenic types (HPV 6/11/16/18).

Treatment of Precancerous Lesions and Cervical Dysplasias—Current Problems—State of the Art

In the state of the art, recurrence rates for all the current excision or surgical treatments used to eliminate/treat precancerous cervical lesions or alterations (dysplasias) are very high, precisely because of the great difficulty in eliminating the main causative agent, that is, the human papilloma virus or HPV.

Given these facts and the remarkable and unprecedented properties of the immunomodulator (proteic aggregate of ammonium and magnesium phospholinoleate-palmitoleate anhydride) demonstrated in the topical use of the compound in the treatment of urinary bladder cancer (Table 4, 5, 6-A, 6-B) and also in the treatment of lesions in the external epithelium, caused by the HPV virus (Table 7), the invention was also tested for topical treatment of cervical dysplasia in women, being associated to the presence of the human papilloma virus (HPV), resulting in a new and unprecedented use for the compound, i.e., the intrauterine topical application of the immunomodulator (proteic aggregate of ammonium and magnesium phospholinoleate-palmitoleate anhydride) for the treatment of the referred disease.

Therefore, in order to demonstrate in practice the effectiveness of the present invention in the treatment of HPV-related cervical dysplasia, an example will be provided of its use in a clinical trial involving patients with chronic HPV infection and cervical dysplasias associated to the infection ranging from mild to moderate, which did not respond satisfactorily to one of the most common standard treatments in the state of the art, that is, a surgical procedure called electrocauterization.

Practical Example of the Use of the Invention for the Topical Treatment of Cervical Lesions Associated to Virus (HPV)—Table 8-A and Table 8-B

In order to demonstrate in practice the effectiveness of the present invention in the topical treatment of HPV-related cervical dysplasia, an example of its use in a clinical trial involving patients with chronic and recurrent HPV infection and cervical dysplasias associated to the infection, ranging from mild to moderate, which did not respond satisfactorily to one of the most common standard treatments in the state of the art, that is, electrocauterization surgery

This practical example is included for illustrative purposes only without intending to limit the field of application or the scope of the invention.

Clinical study: Postsurgical application of the immunomodulator (proteic aggregate of ammonium and magnesium phospholinoleate-palmitoleate anhydride) in 25 women with moderate to severe dysplasia confirmed by laboratory tests (Papanicolaou test) and HPV seropositivity (PCR method).

All patients had previous history of routine surgical procedures for removal of the damaged tissue (electrocauterization) in the months prior to the clinical trial, with recurrence of dysplasia and infection in all the cases.

The patients underwent routine examinations before the clinical trial for evaluation of the degree of dysplasia (Papanicolaou test) and cervical smears were taken for human papilloma virus (HPV) testing and typing.

Of the 25 patients that participated in the clinical trial, 15 were classified as suffering from moderate cervical dysplasia associated to HPV 11, 5 with moderate cervical dysplasia associated to HPV 18, and 5 with severe cervical dysplasia associated to types HPV-16 and HPV-18.

The results of the tests of patients on admission to the trial are shown in Table 8-A.

TABLE 8-A
Classification of the degree of cervical dysplasia and viral
typing performed on admission to the clinical trial (basal)

| Number of | Mild | Moderate | Severe | |
|---|---|---|---|---|
| patients | dysplasia | dysplasia | dysplasia | Type of HPV |
| n = 25 | (**) | (**) | (**) | associated (*) |
| 15 | — | 15 | — | HPV 11 |
| 5 | — | 5 | | HPV 18 |
| 5 | — | — | 5 | HPV 16/18 |

(*)Method for detection and viral typing: PCR - Kit Amplicor (HPV) - Roche Method for detection and classification of cervical dysplasia: Papanicolaou (**)

Periodicity: Collection of cervical smear for the test and classification of dysplasia and HPV were performed in the pretreatment period (basal) and 6 months after the end of the clinical trial.

Characterization of the sample: Patients previously treated with surgery (electrocauterization) and showing signs of recurrent cervical dysplasia, classified by the Papanicolaou test as moderate to severe dysplasias, upon admission to the clinical trial (Table 8-A).

Viral typing: All patients (100%) had at least one viral type considered of high risk for developing malignancy on admission, and five patients showed association of more than one viral type (HPV 16 and HPV 18) (Table 8-A).

Clinical trial design: All patients underwent the same surgical procedure, that is, electrocauterization for removal of the abnormal tissue (dysplasia).

The patients were also given the (proteic aggregate of ammonium and magnesium phospholinoleate-palmitoleate anhydride), topically administered, for this particular purpose in the form of a topical cream (pH 7.0), with a concentration of the active ingredient (proteic aggregate of ammonium and magnesium phospholinoleate-palmitoleate anhydride) of 20% daily applied using a intrauterine applicator for 2 weeks, the first dose beginning 1 day after surgical cauterization (electrocauterization).

Follow-up: All patients were followed monthly through clinical examinations and colposcopy.

Final assessment: the patients were assessed again 180 days after the end of the surgical procedure combined with the applications of the immunomodulator (proteic aggregate of ammonium and magnesium phospholinoleate-palmitoleate anhydride), topically administered, for classification of the type of dysplasia and the possible presence of HPV.

Results: The results are shown in Table 8-B and then discussed.

TABLE 8-B
Classification of the degree of cervical dysplasia and viral
typing after the clinical trial (T: 180 days after the end)

| | Mild | Moderate | Severe | |
|---|---|---|---|---|
| Number of | dysplasia | dysplasia | dysplasia | HPV type |
| patients | (**) | (**) | (**) | associated (*) |
| 20 | — | — | — | — |
| 3 | — | | — | HPV 11 |
| 2 | — | — | | HPV 16/18 |

(*) Method for detection and viral typing: PCR - Kit Amplicor (HPV) - Roche
(**) Method for detection and classification of cervical dysplasia: Papanicolaou

Table 8-B—Discussion of the Results

The topical use of the compound of the present invention (proteic aggregate of ammonium and magnesium phospholinoleate-palmitoleate anhydride) associated to a surgical procedure (electrocauterization) showed remarkable results for the patients who participated in the clinical trial, both regarding cervical dysplasia and the causative agent (HPV). Although the sample is limited in number, it should be noted that all patients had previously undergone the same surgical procedures for removal of the abnormal tissue (cervical dysplasia) and relapsed, that is, 100% of recurrence was observed. The recurrence rate with the use of the invention, that is, associated to surgery (electrocauterization) was 0% (zero percent), i.e. none of the patients showed any sign of dysplasia during the assessment performed 180 days after the end of the clinical trial, although 5 (five) patients still had HPV on the day of assessment after the end of the clinical trial, that is, 180 days later.

Comparatively, the rate of recurrence prior to the beginning of the experiment, reported for these patients, when treated only with surgical methods, was 100% of recurrence for cervical dysplasia and viral presence.

It should be noted that these patients no longer had cervical dysplasia in the assessment period (180 days after the end of the clinical trial), which clearly indicates a direct effect of the invention on cellular change (cervical dysplasia), that is, on cells with potentially precancerous lesions, regardless of the presence of the virus, since the laboratory tests indicated the presence of HPV in 5 (five) patients.

This effect of the invention on the precancerous lesion (cellular dysplasia), regardless of the presence of the virus, has been clearly demonstrated, since for all patients of the clinical trial who were diagnosed with dysplasia ranging from moderate to severe, the dysplasia was completely eliminated after treatment with the invention, despite de presence of the virus in a small percentage, that is, only 5 (five) patients (Table 8-B).

Absolute Novelty of the Invention for the Topical Treatment of Precancerous Lesions Associated to HPV

The use of the immunomodulator (proteic aggregate of ammonium and magnesium phospholinoleate-palmitoleate anhydride) as a component of drug associations or combinations against facultative or strict intracellular pathogens or parasites is described in WO 2009/097670 A1, which includes general antiviral action, once these pathogens are classified as strict intracellular parasites.

However, it can be affirmed that this previous invention (WO2009/097670 A1), or else, the practical use of the immunomodulator (proteic aggregate of ammonium and magnesium phospholinoleate-palmitoleate anhydride) for the treatment of infections caused by intracellular parasites is totally different from the present invention, including the specific treatment of associated precancerous lesions or HPV-related cervical dysplasias.

The previous invention (WO 2009/097670 A1) concerns the use of the immunomodulator (proteic aggregate of ammonium and magnesium phospholinoleate-palmitoleate anhydride), systemically administered with the immunomodulator (proteic aggregate of ammonium and magnesium phospholinoleate-palmitoleate anhydride) injected into the body (intramuscular application) for the treatment of infectious diseases, including those of viral origin.

Finally, the use of the immunomodulator (proteic aggregate of ammonium and magnesium phospholinoleate-palmitoleate anhydride) as adjuvant therapy of cervical dysplasia and infection by HPV is described in WO 2011082458 A1. However, such practical use concerns a different application, that is, the systemic use of the compound of the present invention, which must necessarily be introduced in the patient's body by intramuscular injection.

This means that in the previous requests, that is, in WO 2011082458A1 and WO 2009/097670 A1 the described usefulness or practical effect of the invention depends on the route of administration of the compound (administration by injection) to produce the desired systemic effects.

As for the present invention, it is intended for a completely different purpose, that is, the practical use of the immunomodulator (proteic aggregate of ammonium and magnesium phospholinoleate-palmitoleate anhydride), alone or associated to other non-drug and/or drug treatments also for the treatment of cancer, though by topical administration, that is, directly applied to the damaged tissue, which is completely different from the previous requests, providing several important practical benefits compared to systemic administration, which shall be described in this report.

Comparative Advantages of the Use of Topically Administered Drugs

Any expert with knowledge in the state of the art can easily understand that the topical use, in the case of similar drugs, offers several and significant advantages compared to the systemic use, since the latter depends, for introducing substances into the body, on methods that cause their metabolism (e.g. oral), or else of potentially aggressive methods that cause poor patient compliance, such as the use of subcutaneous and/or intramuscular injections, for example.

Some advantages of the use of topically administered drugs, compared to injectable similar compounds, are cited in a non-exhaustive list, as follows:

A) A lower rate or possibility of occurrence of adverse effects, such as pain and/or muscle inflammation and even less discomfort for patients who use the topical pharmaceutical form, which undeniably leads to greater patient adherence to treatment protocols;

B) Regarding similar drugs, another comparative advantage provided by the topical use is the absolute control of the amounts of drugs to be introduced into the patient's body.

In other words, much larger amounts or concentrations of the active ingredient can be used at the site of the lesion compared to the amounts or concentrations of the same compounds that require systemic administration, for example, oral administration or injection;

Inversely, the topical administration of drugs, also allows the use of smaller amounts of the active ingredient at the site of the lesions, without loss of efficacy, when compared to the amounts or concentrations that can be used with the same compounds, though systemically introduced into the body (orally or by injection).

Or else, the use of drugs capable of topical action allows entire control of the amounts deposed in the body, which cannot be obtained with drugs that require systemic administration, e.g., orally or injected, which necessarily involves their metabolism by the body prior to distribution or deposition in the body tissues, including the damaged tissue.

C) Another comparative advantage of the topical use of the invention is the minimization or even elimination of the occurrence of chemical and/or structural changes in the drug caused by its metabolism in the body. This means that the maximum possible amount of the active ingredient can be used on the site of the lesion or disease, by topical administration.

D) Finally, among some other comparative advantages of the topical use of the invention we also cite: it avoids fluctuation in drug levels due intra patient variations, improves the ability to deliver drug more selectively to a specific site, the avoidance of gastro-intestinal incompatibility, improves physiological and pharmacological response, and improves patient compliance.

Comparative Advantages of the Use of the Invention in the Topical Treatment of Premalignant and Malignant Lesions of the Lining Epithelium.

Considering the information contained in the present report, and also examples of practical use, it can be affirmed that compared to the drugs used in the treatment of cancer affecting the epithelial tissue lining surfaces outside and inside the body, notably urinary bladder cancer, malignant skin and uterine lesions associated to HPV, the present invention, alone and/or in association with other drugs and treatments known in the state of the art, allows significant advances over the state of the art, which also includes the previous use of the immunomodulator proteic aggregate of ammonium and magnesium phospholinoleate-palmitoleate anhydride described in WO 2009/097670 A1 and WO 2011082458 A1) when it depended on systemic administration by injection.

Some benefits and advances allowed by the invention, that is, the topical use of the immunomodulator (proteic aggregate of ammonium and magnesium phospholinoleate-palmitoleate anhydride), considering the state of the art, follow:

A—It has been demonstrated, also with examples of practical use, that the topical administration of the compound of the present invention (proteic aggregate of ammonium and magnesium phospholinoleate-palmitoleate anhydride) has greater therapeutic effectiveness than the most effective immunotherapeutic drug in the closest state of the art for the topical treatment of urinary bladder cancer, which is BCG vaccine (Table 4, Table 6-A, Table 6-B, Table 6-C).

B—It has been shown, also with examples of practical use, that the compound (proteic aggregate of ammonium and magnesium phospholinoleate-palmitoleate anhydride), alone, in topical administration, has wider therapeutic effectiveness than the main chemotherapeutic agents in the state of the art for the treatment of malignant lesions of the epithelial tissue lining inside surfaces, notably in the urinary bladder, in topical application (Table-5)

C—The use of the invention, represented by the isolated use of the compound (proteic aggregate of ammonium and magnesium phospholinoleate-palmitoleate anhydride) in the topical treatment of urinary bladder cancer or associated with other drugs was not accompanied by side effects that are indicative of toxicity, such as hematuria (Table 4, Table 5, Table 6A, Table 6-B, Table 6-C, Table 6-D). On contrary, the hematuria disappeared of all animals treated with the claimed invention (Table 4, Table 5, Table 6A, Table 6-B, Table 6-C).

D) It has also been demonstrated, with examples of practical use, that the use of the invention in combination with chemotherapy and immunotherapy drugs (e.g. BCG), not only maximizes the effects of the compounds in the treatment of malignant lesions in the epithelial tissue lining inside surfaces, notably the urinary bladder, but also contributes significantly to the reduction of the unwanted side effects of such treatments (Table 5, Table 6-A, Table 6-B, Table 6-C, Table 6-D).

E) It has been demonstrated, also with examples of practical use of the invention, that the compound of the invention (proteic aggregate of ammonium and magnesium phospholinoleate-palmitoleate anhydride) can replace the treatments cited, that is, Mitomycin C, Valrubicin, Gemcitabine, Docetaxel and BCG vaccine, in the case of failure or occurrence of toxicity that prevents their continued use (Table 5 and Table -6-A, Table 6-C).

F) It has been demonstrated, also with examples of practical use, that the compound (proteic aggregate of ammonium and magnesium phospholinoleate-palmitoleate anhydride) used after chemical or surgical removal of lesions is effective as a topical adjuvant treatment of premalignant lesions of the epithelial tissue lining surfaces inside and outside the body associated to aggressive agents such as the HPV virus, notably in the skin and in the uterine region (Tables 7 and Table 8-B)

Extrapolation of the Usefulness of the Invention for Tumors Located in Other Luminal Organs, Using Devices for the Application of Drugs

These impressive effects of the use of the compound (proteic aggregate of ammonium and magnesium phospholinoleate-palmitoleate anhydride) in the treatment of cancer, by topical administration in the affected epithelium, illustrated in the examples cited in the present report, that is, topical treatment of precancerous lesions in the epithelium lining external surfaces (skin) associated to HPV demonstrate that the invention can be used for the topical treatment of malignant and premalignant lesions in the epithelial tissue lining outside (skin) and inside (mouth, esophagus, stomach, small intestine, urinary bladder, urethra, vagina, esophagus) surfaces.

Also, the remarkable effects of the use of the compound (proteic aggregate of ammonium and magnesium phospholinoleate-palmitoleate anhydride) in the treatment of cancer, topically administered to the affected epithelial tissue of inside surfaces, illustrated by examples cited in the present report, that is, topical treatment (intravesical) of urinary bladder cancer and of intrauterine lesions associated to HPV demonstrates the efficacy of the invention in the topical treatment of malignant and premalignant lesions in the epithelial tissue lining inside surfaces, notably in the urinary bladder and uterus.

The remarkable effects of the use of the compound (proteic aggregate of ammonium and magnesium phospholinoleate-palmitoleate anhydride) in the treatment of cancer, by topical application on the affected epithelium, illustrated by the experiments cited in the present report, i.e., the topical treatment (intravesical) of urinary bladder cancer, precancerous lesions in the epithelial tissue lining inside and outside surfaces indicate the use of the invention for the treatment of cancer affecting internal body regions, provided these can be reached by devices for administration of topical medication, such as catheters, cannulae, and others.

For the topical treatment of lesions in the epithelial tissue lining inside surfaces, all types of pharmaceutical vehicles known in the state of the art, such as creams, lotions and other vehicles of application and dispersion of drugs can be used for the purposes of topical application of the invention.

In short, all kinds of mechanical or pharmacological devices able to carry or release drugs can be used for the administration of the compound of the invention in the treatment of lesions in the epithelial tissue lining outside surfaces and also inside surfaces of luminal organs such as mouth, esophagus, stomach, small intestine, urethra bladder, vagina and uterus.

Formulations or Pharmaceutical Forms for the Practical Use of the Invention

Three pharmaceutical formulations for the practical use of the invention that were found to be suitable for topical administration of the invention in animal models and in humans and were presented in this report follow.

State of Art

Although some of the beneficial effects of immunomodulators are known when used systemically, the ability to provide therapeutic benefit via local administration of these compounds due its immunologic effects (e.g. BCG) for treatment of a particular condition (e.g. malignant lesions) at a particular location (e.g. urinary bladder, uterus) may be impaired or blocked by a variety of factors.

One aspect of particular relevance for the purposes of the invention is that the carriers used for drug delivery to these particular locations obligatorily may be inert or else, not causes any degradation of the compounds transported as single agents or in combination. Further, carriers used for the purposes of drug delivery may not be able to induces inflammatory changes on the urothelium or aggravate a prior inflammation caused by the disease (e.g. bladder cancer) and/or other treatments (e.g. surgery-TUR or intravesical BCG).

For the purposes of the invention, that is, topical treatment of malignant and premalignant lesions inside and outside the body, a pharmaceutical acceptable carrier comprises only the substances or compositions that are unable to cause degradation of drugs as well as not damages the body's tissues, and finally not modifies or damages the devices used for drug delivery.

For instance, as largely known, therapeutic agents and other compounds that are not soluble in water are often soluble in dimethyl sulfoxide (DMSO). The immunomodulator (proteic aggregate of ammonium and magnesium phospholinoleate-palmitoleate anhydride) used for the purposes of the invention is poorly soluble in aqueous solutions (Specification, Table 6-B). Thereby, DMSO could be thought of as a hypothetical carrier. However, as largely known DMSO at some concentrations may be harmful to tissues and cells. Thereby, DMSO is not a pharmaceutical acceptable carrier taking into account the purposes of the invention.

Election of Sodium Chloride Solution at Concentration of 0.9% (9 g NaCl/Liter) as an Inert Carrier for the Purposes of the Invention

The first formulation comprises essentially the suspension of the compound used for the purposes of the invention in sterile saline solution for injection (sodium chloride solution) at concentration of 0.9% (9 g NaCl/liter) for use or deposition in luminal organs for the treatment of premalignant or malignant lesions of the epithelial tissue lining inside surfaces, through catheters or other devices. One advantage of this formulation is the possibility of its application on any epithelial tissue lining inside surfaces, such as mouth, esophagus, stomach, intestines, urethra, urinary bladder, vagina, and uterus, because saline solution at 0.9% (NaCl 0.9%) is known to be harmless to cellular components.

In this setting, sodium chloride solution at concentration of 0.9% (9 g NaCl/liter) was selected as acceptable pharmaceutical carrier for the purposes of the invention. As largely known by any person skilled in the art, sodium chloride solution at concentration of 0.9% (9 g NaCl/liter) is an isotonic solution. Of note, isotonic solution such as provided by the invention, besides its very low reactivity, is harmless to any cellular components, as well for devices or materials used for the therapeutic purposes (Barabino S. et al. Effects of a 0.9% sodium chloride ophthalmic solution on the ocular surface of symptomatic contact lens wearers. Can J Ophthalmol 2005; 40:45-50).

Thereby, as cited above, the advantage of the formulation—as provided by the invention that uses sodium chloride solution at concentration of 0.9% as pharmaceutically carrier—is the possibility of its use on any epithelial tissue lining inside or outside surfaces, without any risks of injuries. Of note, saline solutions, such as sodium chloride solution at concentration of 0.9% is also the carrier used for intravesical administration of BCG in patients with bladder cancer (For reference: ImmunoCys—Instructions for end users).

The method of treatment provided by the invention make use of the immunomodulator alone or in association with BCG and in some occasions (BCG-refractory disease and BCG-intolerance) make possible its replacement by the first drug. Thereby, the use of sodium chloride solution at concentration of 0.9% as carrier for both compounds make possible the administration of the two drugs in combination or sequentially without risks of drug degradation and more important, not causing or aggravating inflammation on the urothelium.

Last but not least, taking into account that the invention comprises the deployment of active compounds in tissues located inside the body (e.g. BCG, chemotherapy, immunomodulator) the use of devices is obligatory (e.g. catheters, intrauterine devices, pessary, etc.). Thus, an inert carrier for purposes of drug delivery is of paramount importance taking into account that any degradation of materials used to make the devices (e.g. polymers, etc.) is not admissible due the risk of administration of such (degraded) materials to end users during the procedures aiming the drug delivery inside the body (e.g. urinary bladder).

Finally, the definition of amounts of 10 mg/kg and 5 mg/kg of the immunomodulator ( ) to make formulations using sodium chloride solution at concentration of 0.9% as carrier provides sufficient information to differentiate the formulations provided herein over the prior art. To conclude and taking the information above, this formulation is new in the state of art, taking into account this use according the purposes of the invention.

The second formulation, which is also used in clinical practice, mentioned in the present report, comprises essentially the formulation of the proteic aggregate of ammonium and magnesium phospholinoleate-palmitoleate anhydride, with the concentration of the active ingredient ranging from 15% to 30%, in the form of cream, which occurs when the compound is added, dispersed and homogenized to a base or inert carrier, formed by sterile petroleum jelly (CAS number 8009-03-8) and sterile deionized distilled water, using a mixer. The final pH will be adjusted to around 7.

The inventive step involved in the selection of petroleum jelly (CAS number 8009-03-8) for make pharmaceutical formulations is that this carrier, due this very low reactivity, not causes degradation of the compounds dispersed nor causes or aggravate inflammation on the urothelium and finally is not able to modify or damage the materials and devices used for drug delivery. In addition, petroleum jelly have a melting point usually within a few degrees of human body temperature (approximately 37° C. or 99° F.) making this carrier appropriated for making formulations aimed to deploy drugs in surfaces inside and outside the body (Petroleum jelly—Information for users). For these reasons it was selected as an acceptable carrier selected for the purposes of the invention.

The third formulation comprises the use of the compound solubilized with Poloxamer, with remarkable and outstanding practical results described in the present report and in Table 6-B. For this formulation for topical use in the epithelial tissue lining inside or outside surfaces, 0.1 of the immunomodulator (proteic aggregate of ammonium and magnesium phospholinoleate-palmitoleate anhydride) is dissolved (suspended) in 0.2 mL of aqueous solution of Ethylene Oxide/Propylene Oxide Block Copolymer (Polaxamer) with final concentration of 1 mg/ml.

The inventive step involved in the selection of Poloxamers as pharmaceutically acceptable carriers to make compositions for the purposes of the invention is due this class of compounds has been recognized as important biomaterials used for the efficient delivery of drugs and acting to enhance the therapeutic activity of the drugs delivered (Batrakova et al). In addition, Polaxamers are known in the state of art as a valuable component to make thermo-sensitive gels for controlled release of therapeutic proteins and peptides with no known toxicity. The immunomodulator contains protein as well as peptides. Therefore, Polaxamers (also termed “Pluronics”) were selected for make a pharmaceutical composition to be used for the purposes of the invention.

This concentration (1 mg/ml) is the lowest concentration experimentally obtained to produce therapeutic effects with the topical use of the immunomodulator (proteic aggregate of ammonium and magnesium phospholinoleate-palmitoleate anhydride), even using Poloxamers as carriers.

Below the concentration of 1 mg/ml for the immunomodulator (proteic aggregate of ammonium and magnesium phospholinoleate-palmitoleate anhydride), no therapeutic results were experimentally obtained, so far, in the topical treatment of lesions in the epithelial tissue lining inside and outside surfaces of the body.

The use of Poloxamers as carriers for the immunomodulator for the purposes of the invention provides a clear advance over the prior art, because make possible to provide a sharp reduction in the amounts of the immunomodulator when used for the purposes of the invention. As any person skilled in the art may understands, a formulation that allows the use of smaller amounts of drugs required to obtain equivalent therapeutic effects of a greater dosage of the same drug, has undeniable economic advantages, and also in what concerns safety due to the smaller number of substances to be deposed in the body's tissue.

In this setting, a pharmaceutical composition using the immunomodulator (proteic aggregate of ammonium and magnesium phospholinoleate-palmitoleate anhydride) dispersed in Poloxamers (polyethylene-polypropylene-glycol) at final concentration of 1 mg/ml used for purposes of the invention, due the surprisingly results as explained in the specification and Table 6-B, represents a novelty over the closest state of art.

New Experiments and Data—Replacement of BCG and Association of the Immunomodulator With Chemotherapy Agents

To exemplify the usefulness of the claimed invention new experiments were performed and described below. The new data and results are presented only for illustrative purposes to help understanding the novelty, usefulness and advantages of the invention, and by no means intend to limit the scope or the field of application of the present invention.

As previously explained in the specification most episodes of local and systemic toxicity of BCG emerge during induction phase or in the first half year of maintenance therapy. The main cause of delays and interruptions of BCG instillations is its local toxicity, such as localized inflammation leading to cystitis and macroscopic haematuria, which affects up to 75% of patients and leads to a withdrawal from BCG treatment in around 15% of patients. The consequences are deleterious, as the effects of BCG against disease recurrence and progression of bladder cancer are only achieved when maintenance therapy is carried out (van der Meijden A P et al 2003, Hinotsu S et al, 2010, Gan C et al, 2013, Brausi M et al, 2014).

As largely known in the state of art, the use of intravesical BCG is often impaired by the emergence of local and systemic toxicity, leading to interruption in the treatment of high-grade NMIBC patients (van der Meijden A P M et al 2003, Witjes J A et al 2013, Spencer B A et al 2012). Cystitis and haematuria are common side local effects associated with the use of BCG (Witjes J A et al 2013). Pathological changes in the urothelium related to the use of BCG include acute and chronic inflammation similar to those ones seen in tuberculous cystitis (Lopes-Beltran A et al 2002). Irritative bladder symptoms with negative urine culture along the treatment of bladder cancer are classified as BCG induced cystitis (Brausi M et al 2014)

In this setting, the termed BCG intolerance emerges as a condition, which is associated with high risk of disease recurrence and progression to invasive and metastatic disease, leading to death. As only less effective treatments are available for these patients in the closest art, the deployment of new drugs associating a better antitumor effect and lower toxicity than BCG could improve the success rates of bladder cancer treatment, thereby avoiding an early cystectomy.

The interruption of macroscopic haematuria in animals treated with the immunomodulator when used alone and/or in association with other drugs and treatments (Table 6-A, 6-B, 6-C, 6-D, Table 9) represents a very important experimental finding, indicating that the immunomodulator has the surprisingly ability to revert the drug-induced cystitis, an event frequently associated with haematuria and responsible for the most treatment stoppage in the setting of BCG-intolerance (van der Meijden A P et al 2003, Gan C et al 2013, Brausi M et al 2014).

Thereby, an experiment to simulate the replacement of BCG by the immunomodulator, (anhydride) to evaluate interruption of hematuria and therapeutic effects against bladder cancer after the replacement of BCG was carried out in the same animal model (Table 6-C, Group C). In addition, a new association of drugs using smaller dosages was also evaluated in the same animal model using the immunomodulator (anhydride) associated with gemcitabine (Table 6-C Group G) and Docetaxel (Table 6-C, Group I), all drugs used by intravesical via. The results and data of interest are provide below (Table 6-C).

Experimental Design:

In brief, 90 female Fischer 344 rats was induced to bladder cancer using 5 mg/kg of n-methyl-n-nitrosourea (MNU) dissolved in 0.30 ml of sodium citrate, intravesically, with the use of a catheter, for 7 weeks as previously described (Specification, Table 4, and Specification Table 6-A). After treatment with MNU, the animals were divided in nine groups of 10 animals each. All animals were treated by intravesical via using catheter for deployment of drugs into the urinary bladder according the schedules described below. At the end of the experiment all animals was sacrificed and fragments of bladder tissues were collected and processed for histopathological analyses. The criteria for histologic evaluation of lesions inside the bladder was previously described in the specification.

Drugs

BCG=Bacillus Calmette-Guérin

Immunomodulator: Immunomodulator: proteic aggregate of ammonium and magnesium phospholinoleate-palmitoleate anhydride

Experimental Groups, Dosages and Schedules

Group A=n=10—Saline (NaCl 0.9%)—for 14 weeks, intravesical (no MNU)

Group B=n=10—no treatment—followed by 14 weeks

Group C=n=10 —BCG (40 mg or 10 UFC) intravesical—for 14 weeks

Group D=n=10—immunomodulator alone (10 mg/kg), 14 weeks, intravesical

Group E=n=10—BCG (40 mg)/6 weeks, intravesical, (stopped 02 weeks) followed by immunomodulator (5 mg/kg) for 6 weeks, intravesical

Group F=n=10—Gemcitabine (10 mg/kg)/14 weeks, intravesical

Group G=n=10—Gemcitabine (5 mg/kg)/7 weeks, followed by immunomodulator (5 mg/kg) for 7 weeks, intravesical

Group H=n=10—Docetaxel (10 mg/kg)/14 weeks, intravesical

Group I=n=10—Docetaxel (5 mg/kg)/7weeks, followed by immunomodulator (5 mg/kg) for 7 weeks.

Diluents and carriers: All drugs, except docetaxel, were suspended in sterile 0.9% sodium chloride solution for injection (NaCl 0.9%) prior to its intravesical administration in animals. Docetaxel was first added to a recommended diluent (12% w/v ethanol/water diluent) and after then added to an infusion solution containing sodium chloride solution (NaCl 0.9%). The final concentration of docetaxel was adjusted to 10 mg/ml.

TABLE 6-C

| Histological | | | | | | | | | |
|---|---|---|---|---|---|---|---|---|---|
| evaluation | | | | | | | | | |
| and | Group | Group | Group | Group | Group | Group | Group | Group | Group |
| toxicology | A | B | C | D | E | F | G | H | I |
| Flat | — | — | 2 | 4 | 3 | 2 | 2 | 1 | 2 |
| hyperplasia | | | | | | | | | |
| Papillary | — | — | 2 | 2 | 2 | 2 | 2 | 1 | 2 |
| hyperplasia | | | | | | | | | |
| Low grade | — | 1 | 2 | — | — | 2 | 1 | 2 | 1 |
| Intraepithelial | | (10%) | | | | | | | |
| neoplasia | | | | | | | | | |
| High-grade | — | 4 | 3 | — | — | 4 | — | 3 | — |
| intraepithelial | | | | | | | | | |
| carcinoma | | | | | | | | | |
| (carcinoma | | | | | | | | | |
| in situ-Tis) | | | | | | | | | |
| Papillary | — | 4 | 1 | — | 1 | — | 1 | 1 | 2 |
| carcinoma | | | | | | | | | |
| (Ta) | | | | | | | | | |
| Squamous | — | 1 | — | — | — | — | — | 1 | |
| metaplasia | | | | | | | | | |
| Normal | 10 | 0 | 0 | 4 | 4 | — | 4 | 1 | 3 |
| Toxicology: | no | +14 | +14 | No | +in 10 | +in 04 | +in 05 | +10 | +10 |
| (Hematuria) | | weeks | weeks | 14 | first | 14 wk | first | 14 | first 7 |
| | | | | weeks | 7 wk | Absent | 7 wk | weeks | weeks |
| | | | | | Absent | in 06 | Absent | | Absent |
| | | | | | next | 14 wk | next | | next |
| | | | | | 7 wk | | 7 wk | | 7 wk |
| | | | | | | | in 10 | | |

Table 6-C—Results and Discussion

The animals of group A (negative control—treated only with saline 0.9%), as expected, shows no cancerous or precancerous lesions at 14 weeks nor hematuria. However, the animals of group B (positive control-treated only with MNU) shows 90% of malignant lesions (01 low-grade epithelial neoplasia, 04 high-grade carcinoma in situ (Tis), 04 papillary carcinoma) and 10% of premalignant lesions (01 squamous metaplasia). All animals of this group presented hematuria during the experiment, due the inflammatory effects of carcinogen (MNU) plus the carcinogenic process.

The Group treated with BCG intravesical alone (Table 6-C, Group C) showed 40% of success rate (02 papillary hyperplasia, 02 flat hyperplasia). However, in this group, 06 animals presented recurrent/refractory malignant lesions (02 low-grade epithelial neoplasia, 03 high-grade carcinoma in situ (Tis), 01 papillary carcinoma (Ta)).

In addition, all animals (Table 6-D, Group C) presented hematuria during the period of treatment, indicating that tough BCG was effective against cancerous lesions in 4 (40%) of animals, the drug induced cystitis that is also characterized by hematuria, that was present, even for these animals, during all the experiment.

In sharp contrast, the animals of Table 6-C—Group D, that used the immunomodulator alone, shows 100% of success rate (02 papillary hyperplasia, 04 flat hyperplasia, 04 normal). Of note, the hematuria was absent for all animals, indicating an effective action on the carcinogenic process as well as against the inflammatory effects of the carcinogen.

The Group E (Table 6-C, Group E, BCG followed by immunomodulator) shows 90% of success rate (02 papillary hyperplasia, 03 flat hyperplasia, 04 normal) and 10% fail (01 squamous metaplasia). Compared with the effects of intravesical BCG alone (Table 6-C, Group C, 40% of success rate) the results clearly indicate that the association of BCG plus immunomodulator, used by intravesical via, was highly effective for the treatment of malignant lesions inside the urinary bladder.

Notably, the hematuria that was present during the use of BCG (10 animals—first 7 weeks) disappear after the replacement of the BCG by the immunomodulator (none animals—last 7 weeks) as indicated in Table 6-C, Group E. This finding indicates that the immunomodulator when added in the association is able to act against both the cancerous process and the inflammatory effects of BCG on the urothelium (BCG induced-cystitis).

Presence of synergistic effects for the association of drugs were found, because a half dose was used for both BCG and the immunomodulator, resulting in remarkable curative effect (90% of success rate, Table 6-C, Group E).

Taking into account the data, the use of immunomodulator with BCG, clearly indicates that the association of drugs provides synergistic effects against malignant lesions. Of note, the safety profile of intravesical immunomodulator compares favorably to that of the current therapies such as BCG, the best therapy for topical treatment of bladder cancer in the closest art. Thereby, the data indicates that the immunomodulator may replace BCG when local or systemic toxicities (BCG-induced cystitis and BCG-sepsis) emerge in the course of treatments of patients suffering of bladder cancer.

Concerning the use of intravesical gemcitabine (10 mg/kg, intravesical, 14 weeks, Table 6-C, Group F), the results are similar to use of intravesical BCG alone (Table 6-C, Group C), resulting in a success rate of 40% (02 flat hyperplasia, 02 papillary hyperplasia). The rates of failure indicating presence of recurrent/refractory lesions also are similar. Hematuria was present during the period of experiment (14 weeks) in 04 animals, indicating that gemcitabine was not fully effective to act both against the malignant lesions and the inflammatory effects related with the carcinogen and the carcinogenic process.

In contrast, the use of the association of gemcitabine (5 mg/kg, 7 weeks) followed by the immunomodulator (5 mg/kg, 7 weeks) provided synergistic effects with 80% of responders (02 flat hyperplasia, 02 papillary hyperplasia, 04 normal) using a half dose and a half period of time (7 weeks) for both drugs (Table 6-C, Group G). In addition, the hematuria that was present in 05 animals in the first 7 weeks was eliminated in the last 7 remaining weeks of treatment for all animals (Table 6-C, Group G), indicating that thanks the presence of the immunomodulator in the association of drugs (gemcitabine) the drug induced cystitis was eliminated.

The group that used intravesical taxanes (Docetaxel, 10 mg/kg) showed the smaller number of responders (Table 6-C, Group H). Only 20% of animals presented histologic signs of tumoral remission at the end of experiment (01 flat hyperplasia, 01 papillary hyperplasia). Hematuria was present for all animals, even for the responders, during all the experiment (14 weeks). In contrast, when the immunomodulator is used in association (Table 6-C, Group I), again synergistic effects emerges (70% responders, 02 flat hyperplasia, 02 papillary hyperplasia, 03 normal) using smaller doses and a half period of use for both drugs. The hematuria, observed in the first 7 weeks of treatments was absent in all animals after the use of the immunomodulator in association (Table 6-C, Group I).

Experiments on Recurrence and Survival Using the Invention—State of Art—Table 6-D

In the state of art, the majority of noninvasive (superficial) bladder cancer patients are treated with “Bacillus Calmette Guérin (BCG) solutions”, which are administered via the intravesical route. However, such BCG solutions are composed by an attenuated form of the bacterium Mycobacterium tuberculosis, and therefore, exhibiting a poor safety profile. In addition, superficial growing tumors can be removed by transurethral resection (TUR) but the recurrence rate is higher for patients submitted only to surgical procedures.

To attempt to prolong the tumor free intervals after surgical resection, intravesical treatment with Mycobacterium bovis Bacillus Calmette Guérin (BCG), the vaccine strain against tuberculosis infection, is currently used as an adjuvant treatment option after the surgery (TUR). However, the drug and this method of treatment also [symbol] [symbol] [symbol] presents high rates of recurrent and refractory tumors, despite an early response.

Taking into account that the major goals in treating patients with NMIBC are to prevent recurrences of malignant lesions and [symbol] the muscle-invasive progression, [symbol] [symbol] there is in the state of art an urgent medical need [symbol] of effective therapies as options in case of BCG failures. Thereby, to evaluate the use of the invention against recurrence of malignant lesions and survival of animals, a new comparative experiment was performed in 36 Fischer rats induced to bladder cancer by MNU, using BCG (12 animals), the imunomodulador alone (12 animals), and the association of the immunomodulator and BCG (12 animals).

Two weeks after the last dose of MNU, the animals were treated with immunomodulator 10 mg/kg, weekly, for 7 weeks (Table 6-D, Group A) BCG 40 mg/kg (Table 6-D—Group B) weekly for 7 weeks, and association of BCG (40 mg/kg) weekly for 7 weeks and the immunomodulator, 10 mg/kg (Table 6—Group C) weekly for 7 weeks. All drugs were suspended in sterile sodium chloride solution for injection (NaCl 0.9%) prior its intravesical administration.

After the last dose of drugs, the animals were kept without any treatment for 1 month (n=6 animals) and 6 months (n=6 animals) in each group to verify a possible tumor recurrence and the rates of survival. At 1 month and 6 months, six animals of each group were sacrificed and fragments of bladder tissues were collected and processed for histopathological analyses. The criteria for histologic evaluation of malignant and premalignant lesions in the urinary bladder was previously described in the specification.

The periods of 1 and 6 months used for follow-up were selected based on relationship between ages of rats and humans beings described in the scientific literature, according to which each month of rats is considered equivalent to 2.5 years of humans (Sengupta P, 2013, Andreollo N A et al, 2012). The results are provided in the Table 6-D.

TABLE 6-D

| | Follow up for recurrence and survival | |
|---|---|---|
| | 1 month after | 6 months after |
| | treatment | treatment |
| | n = 6 | n = 6 |
| Histopathology | | |
| GROUP A | | |
| Immunomodulator alone | | |
| n = 12 | | |
| Normal | 2 | 2 |
| Flat Hyperplasia | 2 | 1 |
| Papillary Hyperplasia | 2 | 2 |
| Low-grade Intraurothelial | — | 1 |
| Neoplasia | | |
| High-grade Intraurothelial | — | — |
| Neoplasia - Carcinoma in | | |
| situ (pTis) | | |
| Papillary Carcinoma (pTa) | — | — |
| Urothelial carcinoma with | — | — |
| lamina propria invasion | | |
| (pT1) | | |
| GROUP B | | |
| BCG alone | | |
| n = 12 | | |
| Normal | 1 | 1 |
| Flat Hyperplasia | 1 | — |
| Papillary Hyperplasia | 3 | — |
| Low-grade Intraurothelial | 1 | — |
| Neoplasia | | |
| High-grade Intraurothelial | — | 1 |
| Neoplasia - Carcinoma in | | |
| situ (pTis) | | |
| Papillary Carcinoma (pTa) | — | 2 |
| Urothelial carcinoma with | — | 2 |
| lamina propria invasion | | |
| (pT1) | | |
| GROUP C | | |
| BGG plus | | |
| immunomodulator | | |
| n = 12 | | |
| Normal | 1 | 1 |
| Flat Hyperplasia | 2 | 1 |
| Papillary Hyperplasia | 3 | 3 |
| Low-grade Intraurothelial | — | 1 |
| Neoplasia | | |
| High-grade Intraurothelial | — | — |
| Neoplasia - Carcinoma in | | |
| situ (pTis) | | |
| Papillary Carcinoma (pTa) | — | — |
| Urothelial carcinoma with | — | — |
| lamina propria invasion | | |
| (pT1) | | |

Discussion and Conclusions

At 30 days after the end of treatments, all groups shows similar results (100% of responders) concerning absence of malignant or invasive lesions. However, at 180 days, the results differ. The animals that used the immunomodulator alone (Table 6-D, Group A, 6 months after) and BCG in association with the immunomodulator (Table 6-D, Group C, 180 days) showed the best response concerning the absence of malignant cells at the end of experiment. The animals of the Group A (immunomodulator alone, 180 days) shows 83% of responders (02 normal, 01 flat hyperplasia, 02 papillary hyperplasia) and 17% of recurrent or refractory lesions (01 low-grade intraurothelial neoplasia).

Also, the animals of the group C (Table 6-D, Group C, BCG plus immunomodulator) shows 83% of responders at 180 days (01 normal, 01 flat hyperplasia, 03 papillary hyperplasia) and 17% of recurrent or refractory lesions (01 low-grade intraurothelial neoplasia).

The animals that used BCG alone by intravesical via (Table 6-D, Group B), tough presents a good outcome at 30 days, that is 83% of responders (01 normal, 01 flat hyperplasia, 03 papillary hyperplasia) and 17% of recurrent or refractory lesions (01 low-grade intraurothelial neoplasia), at 180 days not presents the same outcome.

At 180 days, the animals of Group B (BCG intravesical alone) shows presence of malignant lesions in 5 animals (83%) and only 01 (17%) animal presenting histologic findings indicative of absence of malignant lesions (Table 6-D, Group B, 180 days).

For animals of Group B, at 180 days after the end of treatment 01 animal presents carcinoma in situ (pTis), 02 animals presents urothelial carcinoma with lamina propria invasion and 02 animals presents papillary carcinoma (pTa).

This important finding (Table 6-D, Group B, 180 days) is totally in agree with evidences from studies in animal models showing that the protective immunity induced by BCG wanes with time (Lamm. et al, 2010). This data from studies using animal models provides the rationale for use of maintenance BCG therapy in human subjects. (Lamm. et al, 2000).

In fact, for treatment of bladder cancer in human subjects, the use of series of intravesical BCG along several years post-surgery (maintenance BCG therapy) is required for the improvement of the success rate of treatments using this drug. Consequently, the number of episodes of drug related toxicity and the costs of treatments for the persons in need thereof, increases.

Conclusions

Concluding, in the state of art of treatment of bladder cancer, despite the use of maintenance therapy using BCG, a large number of patients remains refractory to BCG treatments. Thereby, taking into account the data provided in the specification, any skilled in the art may visualizes without any difficult that the method of treatment provided by the invention represents a clear improvement over the prior art concerning the treatment of superficial malignant and premalignant lesions in the urinary bladder.

Based on experimental data (Table 6-D, Group A and Group C, 180 days) any skilled in the art can conclude that the use of the claimed invention, shows a clear superiority over the best intravesical therapy (BCG) available in the closest art (Table 6-D, Group B, 180 days) concerning the elimination of refractory or relapsing malignant lesions inside the urinary bladder after treatments.

In addition, as indicated by the data of Table 6-D, Group C, 180 days, the immunomodulator may replace or supplement BCG in the setting of BCG-related toxicity that impairs its use as well as for treatment of malignant lesions refractory to BCG (BCG refractory disease). Clearly, the claimed invention provides a new and useful method of treatment and represents a remarkable improvement over the state of art concerning the topical treatment of bladder cancer.

Experiments on Spontaneous Canine Bladder Cancer

Dogs with naturally occurring bladder cancer are of special interest as experimental model because the evolution of spontaneous canine tumors, closely mimic the human biological behavior, including sites and frequency of metastasis, and responses to therapy of human bladder cancer, specifically high-grade invasive urothelial carcinoma, in cellular and molecular features. The presenting clinical signs are also similar between dogs and humans, with hematuria being the most common change noted in both species.

Therefore, they are very useful to make research on bladder cancer, to evaluate new therapies. Knapp D W et al. 2014. Urinary Bladder Cancer in Dogs, a Naturally Occurring Model for Cancer Biology and Drug Development. ILAR Journal, Volume 55, Number 1. Schiffman J D and Breen M. Comparative oncology: what dogs and other species can teach us about humans with cancer. Philos Trans R Soc Lond B Biol Sci. 2015 Jul. 19; 370(1673).

In the state of art, the main systemic drugs used for treatment of spontaneous canine bladder cancer are the nonsteroidal antiinflammatory drugs (NSAID), also known as Cox inhibitors for blocking the activity of the cyclooxygenase (Piroxicam, C₁₅H₁₃N₃O₄S, PubChem 5280452). Piroxicam is a nonsteroidal anti-inflammatory drug previously used to treat arthritis in humans. Taking into account that the main objective of intravesical adjuvant therapy is to avoid or at least retard the evolution of bladder cancer to invasive and metastatic presentations of disease, dogs presenting spontaneous urinary bladder cancer were selected for comparative evaluation of the invention. In these experiments, the aims are to evaluate the antitumoral effects of the immunomodulator with and without surgery and compare it with the main drug therapy available in the state of art (piroxicam).

Number of animals: 20-twenty female dogs (four animals/group) were used in the veterinary clinical trial.

Goals: To evaluate antitumoral effects, progression of disease, survival and presence or not of undesirable side events.

Controls

Major evaluations of each animal consist of physical examination, rectal exam, and abdominal ultrasonography, to look for evidence of enlarged lymph nodes and masses (indicative of metastases).

Results: All animals showed be positive for presence of masses evidenced by abdominal ultrasonography, confined into the urinary bladder, without signs of invasion of nearby structures at the beginning of the experiment.

Biopsies: Fine-needle aspiration biopsy ultrasound-guided was used to collected samples for histologic analyses.

Results of histologic evaluations: All animals showed to be positive for presence of transitional cell carcinoma (TCC) in the urinary bladder.

Follow up studies included clinical examination, complete blood cells count, biochemical analyses, and ultrasonography.

Histologic analysis, by fine-needle aspiration biopsy ultrasound-guided including tumor at the end of treatment was also carried out in due times.

Hematuria: presence or not of macroscopic hematuria was considered as a clinical marker for toxicity and effectivity of treatments.

Due to ethic reasons, the use of negative controls (saline) or placebo is not a viable option in veterinary trials enrolling major species presenting disease.

Abbreviations used:

CTR=Complete therapeutic response (disappearance of all clinical and ultrasound evidence of tumor for a minimum of 30 days after the end of treatment),

PTR=Partial therapeutic response (at least 20% decrease in tumor volume with no new bladder lesions for a minimum of 30 days after the end of treatment),

MTR=Minor therapeutic response (<50% change in tumoral mass with no new bladder lesions for a minimum of 30 days after the end of treatment),

NR: No response,

PGD 1=Progressive disease (>50% change in tumoral mass with new bladder lesions for a minimum of 30 days after the end of treatment),

PGD 2=Progressive disease (>50% change in tumoral mass with new bladder lesions and presence of invasive disease or metastasis for a minimum of 30 days after the end of treatment),

RD: Recurrent disease: Presence of bladder lesions after treatment,

Hp=Presence of hematuria,

Ha=Absence of hematuria.

Compounds

Immunomodulator: proteic aggregate of ammonium and magnesium phospholinoleate-palmitoleate anhydride

Cox Inhibitor: Piroxicam, C15H13N3O4S, PubChem5280452, IUPAC: (8E)-8-[hydroxy-(pyridin-2-ylamino) methylidene]-9-methyl-10,10-dioxo-10λ6-thia-9-azabicyclo[4.4.0]deca-1,3,5-trien-7-one).

Dosages, Procedures, Via of Administration and Schedules

Group A: n=4: Immunomodulator: injected: 5 mg/kg, intramuscular, one intravesical application by week using cystocentesis, for 6 weeks, no previous surgery.

Group B: n=4: Immunomodulator: intravesical: 5 mg/kg, one intravesical application by week using cystocentesis, for 6 weeks, no previous surgery.

Group C: n=4: Immumodulator: intravesical: 5 mg/kg, one application intravesical by week using cystocentesis, for 6 weeks, with previous surgery for ablation of all visible masses (surgery performed using fine needle aspiration ultrasound guided).

Group D: n=4: Cox inhibitor (Piroxicam): n=4: Oral (5 mg/kg/day) for 6 weeks, no surgery.

Group E: n=4: Only surgery (surgery performed using fine needle aspiration ultrasound guided).

The dogs were evaluated for tumoral evolution (by ultrasonography) and deaths at 30, 60, 90 and 180 days after initiation of therapy (Table 9.) Presence or not of macroscopic hematuria was also followed (Table 9).

TABLE 9
Groups and Follow up

| | Dosages | 30 days | 60 days | 90 days | 180 days |
|---|---|---|---|---|---|
| Group A | 5 mg/kg | 02 MTR | 02 MTR | 02 MTR | 02 MTR |
| Immunomod. | | 01 PTR | 01 PTR | 01 PTR | 01 PTR |
| Injected | | 01 NR | 01 NR | 01 NR | 01 death |
| (i.m) | | 01 Hp | 01 Hp | 01 Hp | 03 Ha |
| No surgery | | 03 Ha | 03 Ha | 03 Ha | |
| Group B | 5 mg/kg | 04 CTR | 04 CTR | 03 CTR | 03 CTR |
| Immunomod. | | 04 Ha | 04 Ha | 01 RD | 01 RD |
| Intravesical | | | | 01 Hp | 01 Hp |
| No surgery | | | | 03 Ha | 03 Ha |
| Group C((*)) | 5 mg/kg | 04 CTR | 04 CTR | 04 CTR | 04 CTR |
| Immunomod. | | 04 Ha | 04 Ha | 04 Ha | 04 Ha |
| Intravesical | | | | | |
| After surgery | | | | | |
| Group D | 5 mg/kg | 04 MDR | 02 MTR | 02 MTR | 03 RD |
| Piroxicam | | 03 Ha | 02 RD | 02 RD | 01 death |
| (oral) | | 01 Hp | 02 Hp | 02 Hp | 03 Hp |
| No surgery | | | | 02 Ha | |
| Group E | — | 04 CTR | 02 RD | 02 RD | 02 RD |
| Only surgery | | 02 Ha | 02 MDR | 01 PGD1 | 02 death |
| | | 02 Hp | 04 Hp | 01 PGD2 | 02 Hp |
| | | | | 04 Hp | |

Results and Conclusions: Use of Immunomodulator Alone by Systemic and Intravesical Via and Used by Intravesical Via Associated with Surgery.

The result of the use of the immunomodulator alone by intravesical via (Table 9, Group B) showed positive clinical outcomes. In the first 30 and 60 days, 100% of animals (04 animals) shows no presence of malignant lesions (CTR). However recurrence of tumoral lesions (RD=recurrence of disease) was shown in 01 animal (25%) at 90 days. Notably, 03 animals remained free of malignant lesions (75% CTR) evaluated at 90 and 180 days. Hematuria was absent for 03 animals at 30, 60, 90, but present in 01 animal at 90 days, due the recurrence of lesions. None animal died during the experiment.

In comparison, when the immunomodulator was used alone by systemic via (intramuscular) at the same dosage (Table 9, group A) the effects was smaller. Only 2 animals showed a Minimal Therapeutical Response (MTR=<50% change in tumoral mass without new lesions) and 01 animal showed Partial Therapeutic Response (PTR=at least 20% decrease in tumor volume with no new bladder lesions) at 30 and 60 days. However, 01 animal shows no response (NR) during all treatment. This animal died at 100 days of the beginning of the experiment. Hematuria was present in one animal during all experiment.

One can conclude that the use of the immunomodulator alone by intravesical via (Table 9, Group B) shows a clear superiority over the use of the same compound used alone by systemic via at equivalent dosages in canine bladder cancer (Table 9, Group A). The results are in line with the results of Table 4 (See Table 4, comparison between the Groups D and Group E) and confirms that for treatment of malignant and premalignant lesions located in the lining epithelium inside and outside the body, the best method of treatment is the topical use of active compounds.

The best result concerning the complete elimination of malignant lesions (CTR=Complete therapeutic response was shown using the association of the immunomodulator (proteic aggregate of ammonium and magnesium phospholinoleate-palmitoleate anhydride) and surgery (Group C, Table 9). In this group, 100% of the malignant lesions in the urinary bladder were eliminated in all animals without any sign of recurrence or progression of disease at 30, 60 and 180 days. In line with this finding, none animals showed any sign of hematuria at 130, 60 and 180 days.

However, the use of the immunomodulator (proteic aggregate of ammonium and magnesium phospholinoleate-palmitoleate anhydride) alone by intravesical via also may be considered as an innovative option for treatment of bladder cancer without the use of surgery (TUR—transurethral resection), as indicated by the results of the Table 4 and Table 9 (Table 4-Group D and Table 9-Group B). This surprisingly and innovative practical result provided by the invention make possible the treatment of bladder cancer in people for whom the surgical procedures (e.g. TUR) are not the better option, such as elderly patients presenting age-associated comorbidities or those with non-resectable bladder cancer lesions.

Considering that the surgery (TUR or TURBT) involves general anesthesia or spinal anesthesia and often causes physical discomfort, urinary bleeding and other most serious complications in the post-operatory setting, in an innovative way, the invention as above described also be used to avoid or minimize the number of surgeries and consequently improving the patient's welfare and compliance to treatments. (For reference: Ankur B. et al, 2016. Grading of complications of transurethral resection of bladder tumor using Clavien-Dindo classification system. Indian J Urol. 2016 July-September; 32(3): 232-237. Doi: 10.4103/0970-1591.185104).

Finally, for patients with unresectable bladder cancer lesions, as well for those that are not eligible for cystectomy or refuse this surgical procedure, the invention may be particularly useful to be used as salvage therapy to prolong live and to improve the patient's welfare (Table 4-Group D and Table 9-Group B).

Use of Nonsteroidal Anti-Inflammatory Drug (Piroxicam) Alone

Concerning the use of Piroxicam (Table 9-Group D) as a positive control, tough a good clinical outcome was shown at 30 days (04 animals presenting MTR=Minor Therapeutic Response (<50% change in tumoral mass with no new bladder lesions), the disease returns (RD) at 60 days (02 animals=50%) and was still present in 03 animals (03 animals=75%) at 180 days. One animal dead in the period of 180 days. Again, one can conclude that compounds that acts systemically are not the better method for treatment of bladder cancer when compared with the results of Table 9, Groups B and C that used intravesical application of compounds (e.g. proteic aggregate of ammonium and magnesium phospholinoleate-palmitoleate anhydride) as a method of treatment.

Use of Surgery Without Use of Any Adjuvant Intravesical Therapy

Concerning the use of surgery without the use of intravesical therapy, the comparative results between the groups C-Table 9 (surgery plus immunomodulator) and Group E-Table 9 (only surgery) clearly show that the use of intravesical drugs after surgery is of paramount importance. As known in the state of art, in human subjects with high-grade bladder, the use of surgery alone also not provides satisfactory clinical outcomes for treatment of high-grade tumors.

In fact, the animals of the Group E-Table 9, submitted only to surgery show premature recurrence of disease (RD) at 60 days (02 animals), progression of cancer to invasive and metastatic forms in 2 animals (01 PD1 and 01 PD2) at 90 days and 02 deaths due cancer at 180 days. Hematuria, which is consider a clinical sign of presence of bladder cancer (See Knapp D W et al, 2014 cited above), was present in all animals at 60 days and beyond.

Recurrence and Progression of Disease

Again, the claimed invention provides impressive effects against the bladder cancer and the recurrence of malignant lesions and progression of disease. The most of animals of the groups that use the invention by intravesical via (Group B-Table 9 Group C-Table 9) not shows recurrence of lesions nor progression of disease of treated animals at 30, 60, 90 and 180 days. Only one animal of Group B (Table 9-Group B) shows recurrence of disease and presence of hematuria at 90 and 180 days after the end of treatment, despite a good clinical result at 30 days after treatment.

In sharp contrast, the groups that not used the invention according the method of use (Group A-Table 9, Group D-Table 9, Group E-Table 9) show 01 case of no-response to therapy and death (Group A=01 NR+01 death), 02 cases of recurrence of disease and 01 death (Group D=02 RD+01 death) and 02 cases of recurrence of disease plus progression of disease and 02 deaths (Group E=02 RD+01 PGD 1+01 PGD 2+02 deaths) respectively.

Table 9—Conclusions

Taking into account that in the state of art, the main objective of intravesical therapy after surgery, is to avoid or at least retard the recurrence of malignant and premalignant lesions and evolution of bladder cancer to invasive and metastatic presentations of disease, which is not satisfactorily achieved by the current topical treatments, clearly the invention represents a remarkable advance over the prior art. (Table 9-Groups B and C).

Additional Information on Future Uses of the Invention Alone or Associated to Other Drugs and Non-Drug Therapies

For the purposes of the present invention, the immunomodulator (proteic aggregate of ammonium and magnesium phospholinoleate-palmitoleate anhydride), can be used alone, for topical application and with other therapeutic modalities, in combination or association of treatments, also topically, as illustrated in the present report. The other active substances for possible use with the invention, in association or combination with compounds, may vary in species, type, quality and quantity, according to the specific requirements of the disease.

The other active substances, with activity against the several types of malignant and premalignant lesions and also the causative agents, such as the HPV virus, are cited in the present report, only as specific examples of the different classes of products in the current state of the art, such as antineoplastic agents, chemotherapeutic and immunotherapeutic drugs, in a non-exhaustive classification or enumeration.

Other substances and therapies with topical action against cancer not specifically cited in the present report as known in the state of the art, or to be discovered and made available for usage according to the purposes of the present invention, can be used, as long as the immunomodulator (proteic aggregate of ammonium and magnesium phospholinoleate-palmitoleate anhydride) is used or maintained in the association or combination, for topical use, as widely described in this report, for it to continue to produce the desired effects.

Also, in some particular situations, as in the event of cancer affecting the epithelial tissue lining inside and outside surfaces, and also located in other organs that are not part of the epithelial tissue, other substances and therapies active against cancer, with systemic action and possibly known in the state of the art, or to be discovered and made available, can be used, as long as the immunomodulator (proteic aggregate of ammonium and magnesium phospholinoleate-palmitoleate anhydride) is used or maintained in the association or combination, for topical use, as widely described in this report, for it to continue to produce the desired effects.

Compound Used for the Purposes of the Present Invention

The present invention uses a compound or immunomodulator known in the state of the art and called proteic aggregate of ammonium and magnesium phospholinoleate-palmitoleate anhydride, originally reported in PI-0305373-3 US 2006/0093628 A1, EP 1529784 A1, though used in the present invention in a way that is entirely different from all the previous applications of the product described so far in the state of the art, with the advantages associated to this specific form of use.

This compound now topically administered for the purposes of the present invention is characterized as proteic aggregate of ammonium and magnesium phospholinoleate-palmitoleate anhydride, with molecular weight 320.000 Dalton (320 KDa), being previously described in PI-0305373-3 US 2006/0093628 A1, EP 1529784 A1.

This compound used in the present invention, has shown in chemical analysis the presence of 11.6±4.0% of total lipids, 22.7±5.0% of palmitoleic acid, 42.9±2.0% of linoleic acid and 32.0±3.0% of oxidated linoleic acid, 20.1±0.9% of magnesium ions, 10.0±3.3% of ammonium ions, 45.2±2.7% of phosphate and 0.49±0.01% of proteins, according to PI-0305373-3 US 2006/0093628 A1, EP 1529784 A1.

The aminoacid distribution in the protein is: Asp 7.19%; Thr 3.56%; Ser 7.56%; Glu 8.53%; Pro 0.5%; Gly 9.69%; Ala 7.46%; Val 1.0%; Met 4.38%, Isoleu 2.54%, Leu 3.03%, Tyr 0.5%, Phe 1.0%, His 2.83%; Lys 3.56%, Trp 1.3% and Arg 35.2%, according to PI-0305373-3 US 2006/0093628 A1, EP 1529784 A1.

The method of production of the immunomodulator (proteic aggregate of ammonium and magnesium phospholinoleate-palmitoleate anhydride), used for the purposes of the present invention is equally contained and reported in the state of the art in PI -0305373-3 US 2006/0093628 A1, EP 1529784 A1, comprising the use of the Aspergillus oryzae fungus (A. oryzae) in appropriate culture medium, resulting in the immnomodulator specifically selected to be used for the purposes of the present invention.

For additional information related to access to the compound used in the present invention, mode of use, biological properties and other relevant information, consult PI-0305373-3 US 2006/0093628 A1, EP 1529784 A1, U.S. Pat. No. 8,889,153B2).

Without the need for additional information, any person skilled in the art, based only on information and explanations contained in the present report, can understand and use the invention to its fully extent.

Although the invention is intended for human use, other animal species can benefit from its therapeutic properties.

1. A method of topical treatment of malignant or premalignant lesions located in epithelial tissue internal and external, preferably in the urinary bladder, skin and uterus. comprising: (a) an immunomodulator, wherein the immunomodulator is a proteic aggregate of ammonium and magnesium phospholinoleate-palmitoleate anhydride, having of 11.6±4.0% of total lipids, 22.7±5.0% of palmitoleic acid, 42.9±2.0% of linoleic acid, 32.0±3.0% of oxidated linoleic acid, 20.1±0.9% of magnesium ions, 10.0±3.3% of ammonium ions, 45.2±2.7% of phosphate, and 0.49±0.01% of proteins, used alone or in association or combination and,
(b) at least one anti-cancer agent or treatment suitable for treating said malignant or premalignant lesions, said agent providing synergistic effects without additional toxicity when used with the immunomodulator

2. The method according to claim 1, wherein the at least one anti-cancer agent is selected from the group consisting of Bacillus Calmette-Guérin-BCG, mitomycin, valrubicin, thiotepa, doxorubicin, cisplatin, gemcitabine, docetaxel, podophyllin, podophyllotoxin and combinations thereof.

3. The method according to claim 1, wherein the treatment is selected from the group consisting of: surgical procedures (surgery, cryosurgery, electrocauterization, surgery associated to polarized light or laser with the use of photosensitizing substances, removal of lesions by chemical abrasion, removal of lesions by electrocauterization, endoscopic ablation, endoscopic radiofrequency, localized radiotherapy, and combinations thereof.

4. The method according to claim 1, wherein the amino acid content in the proteic aggregate of ammonium and magnesium phospholinoleate-palmitoleate anhydride is: Asp 7.19%, Thr 3.56%, Ser 7.56%, Glu 8.53%, Pro 0.5%, Gly 9.69%, Ala 7.46%,Val 1.0%, Met 4.38%, Isoleu 2.54%, Leu 3.03%, Tyr 0.5%, Phe 1.0%, His 2.83%, Lys 3.56%,Trp 1.3%, and Arg 35.2%.

5. A method for topical treatment of malignant or premalignant lesions affecting superficial internal epithelial tissues and superficial external epithelial tissues, the method comprising administering a therapeutically effective amount of the compound of claim 1 to an organism, wherein the anti-cancer agent or treatment is suitable for treating the target lesion.

6. The method according to claim 5, wherein the immunomodulator and anti-cancer agents or treatments are administered to the subject in need thereof jointly, simultaneously, consecutively or sequentially.

7. A method for topical treatment of malignant or premalignant lesions affecting superficial internal epithelial tissues and superficial external epithelial tissues the method comprising administering a therapeutically effective amount of the compound of claim 1 to an organism, wherein the cancerous or precancerous lesions comprises lesions of the skin, mouth, esophagus, stomach, gross intestine, small intestine, penis, urinary bladder, vagina, and uterus.

8. A method for topical treatment of malignant or premalignant lesions affecting superficial internal epithelial tissues and superficial external epithelial tissues follow excision of malignant or premalignant lesions, the method comprising administering in a subject in need thereof a therapeutically effective amount of the compound of claim 1.

9. The method of claim 7, wherein the excision of lesions comprises chemical abrasion and surgical procedures.

10. A method for treatment of unresectable bladder cancer, the method comprising administering in a subject in need thereof a therapeutically effective amount of the compound of claim 1.

11. A method of palliative treatment of cancer affecting superficial internal epithelial tissues and superficial external epithelial tissues the method comprising the topical administration to a subject in need thereof of a therapeutically effective amount of the compound of claim 1.

12. A method for replacement of Bacillus Calmette-Guérin-BCG, mitomycin, valrubicin, thiotepa, doxorubicin, cisplatin, gemcitabine, docetaxel, podophyllin, podophyllotoxin and combinations thereof in the topical treatment of malignant and premalignant lesions affecting the urinary bladder comprising administering to a subject in need thereof an effective amount of the compound according to claim 1.

13. A method of treatment of cystitis, drug-induced cystitis and hematuria occurring in bladder cancer and treatments, the method comprising administering to a subject in need thereof an effective amount of the compound according to claim 1.

14. The method according to claim 1, wherein said synergistic effects are selected from the group consisting of potentiating therapeutic effects, using smaller doses of anti-cancer agents, diminution of undesirable side effects, elimination of haematuria.

15. A pharmaceutical composition for topical use comprising the compound of claim 1 and a pharmaceutically acceptable carrier.

16. The pharmaceutical composition according to claim 15, further comprising a component selected from the group consisting of sodium chloride solution at concentration of 0.9% (NaCl 9 g/liter), Poloxamers (Pluronics), excipients, a suspension, a transporter, stabilizers and combinations thereof.

18. The pharmaceutical composition according to claim 15, wherein the pharmaceutical composition is a topically applicable pharmaceutical composition selected from the group consisting of isotonic solutions, gel, hydrogel, creams and combinations thereof.

19. The pharmaceutical composition according to claim 15, wherein the pharmaceutical composition is a solution for treatment of bladder cancer comprising a concentration of one part of the immunomodulator, dissolved in one part of aqueous solutions of polaxamers.

20. The pharmaceutical composition according to claim 15, wherein the pharmaceutical composition is a topical cream for treatment of genital precancerous lesions in man caused by HPV comprising sterile petroleum jelly and sterile distilled deionized water with a concentration of 15% of the immunomodulator.

21. The pharmaceutical composition according to claim 15, wherein the pharmaceutical composition is a topical cream for treatment of genital cervical precancerous lesions caused by HPV in woman comprising sterile petroleum jelly and sterile distilled deionized water with a concentration of 20% of the immunomodulator.

22. The pharmaceutical composition according to claim 15, wherein the pharmaceutical composition comprising the immunomodulator using dosages of 5 mg and 10 mg suspended in a sodium chloride solution, wherein the concentration of the sodium chloride solution is 0.9% (NaCl 9 g/liter).

23. The method of claim 8, wherein the excision of lesions comprises chemical abrasion and surgical procedures.
